SEQUENCE LISTING (8 sequences)

SEQ ID NO: 1 - DNA, 18 bp, Artificial Sequence
  Synthetic primer
gcgcagtcagcaaatgaa

SEQ ID NO: 2 - DNA, 20 bp, Artificial Sequence
  Synthetic primer
tgtcaacatggggaacaaca

SEQ ID NO: 3 - DNA, 23 bp, Artificial Sequence
  Synthetic primer
cctgagcaggatggagaattaca

SEQ ID NO: 4 - DNA, 19 bp, Artificial Sequence
  Synthetic primer
tccagaacatgccgcagag

SEQ ID NO: 5 - DNA, 22 bp, Artificial Sequence
  Synthetic primer
cgactccgaaggtgttaactgt

SEQ ID NO: 6 - DNA, 22 bp, Artificial Sequence
  Synthetic primer
gaggtccttgatcttctgttgg

SEQ ID NO: 7 - DNA, 22 bp, Artificial Sequence
  Synthetic primer
caggcattgctgacaggatgca

SEQ ID NO: 8 - DNA, 22 bp, Artificial Sequence
  Synthetic primer
ggccaggatggagccaccgatc

(19) United States
(12) Patent Application Publication
(10) Pub. No.: US 2010/0297123 A1
(43) Pub. Date: Nov. 25, 2010

(54) COMBINATION THERAPY TO INHIBIT T CELL EFFECTOR FUNCTION

(76) Inventors: Fathman C. Garrison, Stanford, CA (US); Jack T. Lin, San Francisco, CA (US)

Correspondence Address: Stanford University Office of Technology Licensing;Bozicevic, Field & Francis LLP, 1900 University Avenue, Suite 200, East Palo Alto, CA, 94303, US

(21) Appl. No.: 12/781,709

(22) Filed: May 17, 2010

Related U.S. Application Data
(60) Provisional application No. 61/178,859, filed on May 15, 2009.

(51) Int. Cl. A61K 39/395; A61K 31/439; A61P 37/06; C12N 5/02; C12N 5/071

(52) U.S. Cl. 424/134.1; 514/291; 424/158.1; 435/375; 435/372.3

(57) ABSTRACT

Methods and compositions are provided for inhibiting the responsiveness of CD4⁺ T cells that have been sensitized to antigen, i.e. CD4⁺ memory, or effector T cells. It is shown herein that such effector T cells will fail to proliferate in response to antigen when inhibited by two compounds, which act on two distinct signaling pathways, the mTOR pathway and the MAPK (MEK) pathway. A synergistic combination of inhibitors is required to achieve effector T cell inactivation because a single inhibitor can provide for inactivation of naïve CD4⁺ T cells, but will not by itself inactivate the sensitized effector T cells.

### GOVERNMENT RIGHTS

This invention was made with Government support under contracts NIHCA65237 awarded by the National Institutes of Health. The government has certain rights in this invention

### INTRODUCTION

Antigenic stimulation of naïve T cells results in differentiation to effector and memory cells, or alternatively in anergy and apoptosis. In order to achieve this initial activation a number of coordinate signals are required, including activation of the T cell receptor (TCR) and CD3 together with a second set of signals delivered by costimulatory receptors. Coreceptors include CD28, cytotoxic T lymphocyte antigen 4 (CTLA4), inducible T cell costimulator (ICOS), programmed cell death protein 1 (PD-1) and CD7, and can function as costimulators (CD28, ICOS and CD7) or corepressors (CTLA4, PD-1) of T cell activation. The scientific literature reflects a long debate on the question whether the TCR and the coreceptors induce separate signal pathways or whether the signaling routes employed by both receptor systems are entirely overlapping.

However, the unwanted immune responses that occur in autoimmune disease and transplant rejection often reflect an ongoing T cell response, which may include activation of naïve T cells, but also includes to a large extent the activation of sensitized T cells, particularly CD4⁺ T cells. In autoimmune disease and graft rejection the problem is an immune response to tissue antigens, and the goal is to downregulate the response to avoid damage to the tissues or disruption of their function. From the point of view of management, the single most important difference between allograft rejection and autoimmunity is that allografts are a deliberate surgical intervention and the immune response to them can be foreseen, whereas autoimmune responses are not detected until they are already established. Effective treatment of an established immune response is much harder to achieve than prevention of a response before it has had a chance to develop, and autoimmune diseases are generally harder to control than a de novo immune response to an allograft. The relative difficulty of suppressing established immune responses is seen in animal models of autoimmunity, in which methods able to prevent the induction of autoimmune disease generally fail to halt established disease.

For example, corticosteroids alter expression of many different genes, and have a broad spectrum of activity, including anti-inflammatory activity. However, there are also many adverse effects, including fluid retention, weight gain, diabetes, bone mineral loss, and thinning of the skin. The use of corticosteroids to control disease requires a careful balance between helping the patient by reducing the inflammatory manifestations of disease and avoiding harm from the toxic side-effects of the drug. For this reason, corticosteroids used in transplant recipients and to treat inflammatory autoimmune and allergic disease are often administered in combination with other drugs in an effort to keep the dose and toxic effects to a minimum.

Cytotoxic drugs kill all proliferating cells and therefore indiscriminately affect all types of activated lymphocyte and any other cell that is dividing. Cyclosporin A, tacrolimus, and rapamycin are more selective and find use in preventing graft rejection, however autoimmune responses are already well established at the time of diagnosis and, in consequence, are much more difficult to suppress. They are therefore less responsive to the immunosuppressive drugs and, for that reason, are usually controlled only with a combination of corticosteroids and cytotoxic drugs.

Tolerance mechanisms play an important role in preventing unwanted immune responses including autoimmunity. T cells are rendered tolerant to self through a combined system involving central and peripheral tolerance. Among several mechanisms to ensure peripheral tolerance is anergy, a state of unresponsiveness induced in CD4 T cells upon activation in the absence of costimulatory signals. In addition to naive CD4 TCR binding to antigenic peptide in the context of MHC, CD28 binding to B7 provided on mature APC allows IL-2 production, a necessary component of naive CD4 T cell activation. The necessity for naive CD4 T cells to receive costimulation and signaling through the IL-2R in addition to TCR ligation serves to create a threshold within the peripheral immune system that both ensures the continued survival and sentry functions of the T cells while also maintaining an immune environment free from autoimmunity.

Some clues to maintenance of the T cell resting state have been found in members of the E3 ubiquitin ligase family, which have been demonstrated to be important molecular mediators of T cell anergy and peripheral tolerance. The ubiquitination process requires the E1 enzyme to activate ubiquitin, an E2 enzyme to act as a transferase, and an E3 ligase to direct substrate specificity for ubiquitination. The E3 ubiquitin ligases Cbl-b, Itch, and gene related to anergy in lymphocyte (GRAIL) have all been described as playing a functional role in T cell anergy. GRAIL was first detected during the induction of anergy in CD4 T cell clones. These and subsequent experiments demonstrated that GRAIL expression rendered the CD4 T cells anergic as measured by impaired proliferation and IL-2 production. Recently, Rho guanine dissociation inhibitor, CD40L, and multiple members of the tetraspanin family have been identified as GRAIL substrates. Otubain-1 (Otub1), a deubiquitinating enzyme, was initially identified as a binding partner and subsequently as an epistatic regulator that destabilized GRAIL protein by allowing autoubiquitinated GRAIL to become degraded in the proteosome.

Tolerance or clonal anergy, defined as the inability of T cells expressing the appropriate clonotypic TCR to respond to antigen presented in otherwise stimulatory conditions, is one means by which auto-destructive immune responses are avoided. Tolerance is known to be an active process wherein TCR signals are propagated intracellularly and then aborted before IL-2 gene expression is achieved. Most tolerant T cells in mice are believed to have a memory (CD45RB) phenotype and express CD44 (pgp-1), suggesting that they can recirculate and respond to local antigenic challenge. Further, they are comprised primarily of T cells that recognize immunodominant determinants of the antigens to which they are tolerant, suggesting that they might compete with immunocompetent T cells that recognize the same determinants. The molecular basis for tolerance is incompletely understood, but in tolerant cells, TCR signals are unable to activate the proto-oncogene Ras, a small G protein that controls the activation of the major family of mitogenic kinases, the MAP kinases.

Since the induction of T cell anergy requires active signaling and new protein synthesis, it is believed that the establishment of the phenotype results in long lasting changes in the pattern of gene expression when compared to naïve cells or effector T cells. In the T cell compartment, GRAIL is expressed primarily in anergy-induced CD4+ T cells. Forced expression of GRAIL inhibits IL-2 transcription even in the presence of full costimulation, and therefore, mimics anergy.

There is a therapeutic interest in understanding the mechanisms that underlie anergy. Loss of anergy in T cells that recognize self-antigens can lead to autoimmune diseases such as insulin dependent diabetes, rheumatoid arthritis, and multiple sclerosis. Conversely, inappropriate anergy may be associated with cancer, where the body fails to mount a response to tumor antigens. The further identification to induce T cell non-responsiveness is therefore of great clinical and scientific interest.

### Publications

Anandasabapathy et al. (2003) Immunity 18(4):535-47 describe expression of GRAIL in anergic CD4+ T cells. Soares et al. (2004) Nat Immunol 5:45-54 describe two isoforms of otubain 1 that regulate T cell anergy via GRAIL. The otubain sequence is described by Balakirev (2003) EMBO Rep. 4(5):517-22. Sequences of known transcripts, and partial sequences, may be found, for example, at Genbank, accession numbers AA679586; AA679586, AA679586; AA679586; AV729441; B1770459; AK098029; AA679586; AK000120; BG818896; R84586; AK091830; and BU859734. The deubiquitinating enzyme UBPY, also known as USP8, is described by Naviglio et al. (1998) EMBO J. 17:3241-3250. Its interaction with Ras-GRF1 is described by Gnesutta et al. (2001) J. Biol. Chem. 276:39448-39454. The interaction of UBPY with Hrs binding protein is described by Kato et al. (2000) J. Biol. Chem. 275:37481-37487.

Neel and Mohi, U.S. published application 20060094674 disclose cancer treatments with a combination of mTOR inhibitor and tyrosine kinase inhibitor.

### SUMMARY OF THE INVENTION

Methods and compositions are provided for inhibiting the responsiveness of CD4⁺ T cells that have been sensitized to antigen, i.e. CD4⁺ memory, or effector T cells. Such T cells are not naïve, i.e. they have been previously activated by antigen. It is shown herein that such sensitized T cells will fail to proliferate in response to antigen when inhibited in two distinct signaling pathways, the mTOR pathway and the MAPK (MEK) pathway. A synergistic combination of inhibitors is required to achieve T cell inactivation because a single inhibitor can provide for inactivation of naïve CD4⁺ T cells, but will not by itself inactivate the sensitized effector T cells.

These activities are relevant in autoimmune disease and immune function, including the regulation of tolerance for example with respect to transplantation recipients. In vitro models and in vivo animal models are also of interest.

### BRIEF DESCRIPTION OF THE DRAWINGS

FIG. 1. GRAIL expression in naive CD4 T cells. A, GRAIL and β-actin immunoblots of Qa-2+ or Qa-2− CD4 single-positive thymocytes from naive BALB/c mice. B, GRAIL and GAPDH immunoblots of CD4 T cells from naive BALB/c mice at indicated hours of bead stimulation (anti-CD3/28). GRAIL-transfected Jurkat cells were used as a positive control (+). Data are representative of more than three experiments with similar results.

FIG. 2. Sustained GRAIL expression following CD4 T cell stimulation diminishes proliferation. A, GRAIL and β-actin immunoblots of CD4 T cells from naive BALB/c mice following retroviral transduction in vitro with GRAIL-GFP or control (vector)-GFP. Protein lysates were made from sorted GFP+ cells from both transduced populations after 24 h of stimulation (anti-CD3/28). B, Proliferation assay of bead stimulated (anti-CD3/28) CD4 T cells retrovirally transduced as in A and sorted for GFP+ cells, without (−) or with (+) stimulation (anti-CD3/28). Error bars indicate SD of triplicates. Measured as counts per minute. Data are representative of two experiments with similar results.

FIG. 3. CD28 costimulation is necessary for GRAIL down-regulation. A, Proliferation assay of APC and peptide OVA (pOVA) stimulated DO11 CD4 T cells isolated from naive DO11 CD28^{+/+} (WT DO11) or DO11 CD28^{-/-} (CD28-DO11) mice. Error bars indicate SD of triplicates. Measured as counts per minute. Data are representative of three experiments with similar results. B, IL-2 ELISA of supernatants collected from DO11 CD4 T cells as in A after 24 h of stimulation (APC/pOVA), measured as ng/ml (n.d., not detected). Error bars indicate SD of triplicates. C, Phospho-STAT5 (Tyr^{694/699}) and total STAT5 immunoblots of re-sorted DO11 CD4 T cells as in A after 48 h of stimulation (APC/pOVA). Data are representative of two experiments with similar results. D, GRAIL and β-actin immunoblots of re-sorted DO11 CD4 T cells as in A after 48 h of stimulation (APC/pOVA). Numbers below blots indicate relative densitometry levels for GRAIL. Data are representative of two experiments with similar results.

FIG. 4. IL-2R signaling down-regulates GRAIL. A, Proliferation assay of CD4 T cells isolated from naive BALB/c mice with (+) or without (−) bead stimulation (anti-CD3/28), including anti-IL-2 Ab (αIL-2) condition. Error bars indicate SD of triplicates. Measured as counts per minute. B, Phospho-STAT5 (Tyr694/699), total STAT5, phospho-Akt (Ser473), and total Akt immunoblots of CD4 T cells as in A after 48 h of bead stimulation. Data are representative of three experiments with similar results. C, Phospho-S6K1 (Thr⁴²¹/Ser⁴²⁴), total S6K1, phospho-4E-BP1 (Thr^{37/46}), and total 4E-BP1 immunoblots of CD4 T cells as in A after 48 h of bead stimulation. On total 4E-BP1 immunoblot, top arrow indicates hyperphosphorylated form and bottom arrow indicates hypophosphorylated form. Data are representative of three experiments with similar results. D, GRAIL and β-actin immunoblots of CD4 T cells as in A, ex vivo (0) or after 48 h of bead stimulation. Numbers below blots indicate relative densitometry levels for GRAIL. Data are representative of three experiments with similar results.

FIG. 5. mTOR inhibition maintains GRAIL expression. A, Phospho-S6K1 (Thr⁵²¹/Ser⁴²⁴), total S6K1, phospho-4E-BP1 (Thr^{37/46}), and total 4E-BP1 immunoblots of CD4 T cells isolated from naive BALB/c mice ex vivo (0), or after 48 h of bead stimulation (anti-CD3/28), including rapamycin (RAPA). On total 4E-BP1 immunoblot, top arrow indicates hyperphosphorylated form and bottom arrow indicates hypophosphorylated form. Data are representative of more than three experiments with similar results. B, Otub-1 (left panel) or GRAIL (right panel) expression levels of CD4 T cells as in A, ex vivo (0) or after 24 h bead stimulation (anti-CD3/28), including RAPA. Otub-1 or GRAIL expression levels were normalized to β-actin expression levels. Error bars indicate SD of triplicates. Data are representative of two experiments with similar results. C, Proliferation assay of CD4 T cells as in A, without (−) or with (+) bead stimulation (anti-CD3/28), including RAPA. Error bars indicate SD of triplicates. Measured as counts per minute. Data are representative of three experiments with similar results. D, Cyclin D3, Kip1/p27, and β-actin immunoblots of CD4 T cells as in A after 48 h of bead stimulation (anti-CD3/28), including RAPA. Data are representative of three experiments with similar results. E, GRAIL, Otub1, and β-actin immunoblots of CD4 T cells as in A, ex vivo (0), or after 48 h of bead stimulation (anti-CD3/28), with RAPA. Numbers below blots indicate relative densitometry levels for GRAIL or Otub1. Data are representative of three experiments with similar results.

FIG. 6. IL-2R signaling requires mTOR activation to regulate GRAIL. A, IL-2 expression levels of CD4 T cells isolated from naive BALB/c mice and RNA harvested from ex vivo (0), or after 24 h bead stimulation (anti-CD3/28), including Rapamycin (RAPA). IL-2 expression levels were normalized to β-actin expression levels. Error bars indicate SD of triplicates. Data are representative of two experiments with similar results. B, IL-2 ELISA of supernatants collected from CD4 T cells as in A, unstimulated (−) or after 24 h of bead stimulation, including RAPA, measured as ng/ml. Error bars indicate SD of triplicates. C, CD25 cell surface staining by flow cytometry of CD4 T cells as in A, unstimulated (−), or after 24 h of bead stimulation, including RAPA and IL-2. Numbers indicate percent CD25 positive. D, Proliferation assay of CD4 T cells as in A, without (−) or with (+) bead stimulation (anti-CD3/28), including RAPA. Error bars indicate SD of triplicates. Measured as counts per minute. Data are representative of three experiments with similar results. E, Phospho-STAT5 (Tyr^{694/699}), total STAT5, phospho-Akt (Ser⁴⁷³), total Akt, phospho-S6K1 (Thr⁴²¹/Ser⁴²⁴) total S6K1, phospho-4E-BP1 (Thr^{37/46}), total 4E-BP1, GRAIL, Otub1, and β-actin immunoblots of CD4 T cells as in A, after 48 of bead stimulation (anti-CD3/28), including RAPA and IL-2. On total 4E-BP1 immunoblot, top arrow indicates hyperphosphorylated form and bottom arrow indicates hypophosphorylated form. Numbers below blots indicate relative densitometry levels for GRAIL or Otub1.

FIG. 7. Human naive CD4 T cells down-regulate GRAIL through CD28 costimulation. A, GRAIL, Otub1, and β-actin immunoblots of human naive CD4+CD45RA+ ex vivo (0), or after 48 h of bead stimulation (anti-CD3/28). Data are representative of three experiments from different donors with similar results. B, Proliferation assay of CD4 T cells as in A, without (−) or with (+) stimulation (anti-CD3/APC), including CTLA4-Ig and anti-CD28. Error bars indicate SD of triplicates. Measured as counts per minute. Data are representative of two experiments from different donors with similar results. C, Cyclin D3, Kip1/p27, and β-actin immunoblots of CD4 T cells as in A, after 48 h of stimulation (anti-CD3/APC), including CTLA4-Ig and anti-CD28 (a28). Data are representative of two experiments from different donors with similar results. D, Phospho-STAT5 (Tyr^{694/699}) and total STAT5 immunoblots of CD4 T cells as in A, after 48 h of stimulation (anti-CD3/APC), including CTLA4-Ig and anti-CD28 (a28). Data are representative of two experiments from different donors with similar results. E, GRAIL, Otub1, and β-actin immunoblots of CD4 T cells as in A, after 48 h of stimulation (anti-CD3/APC), including CTLA4-Ig and anti-CD28 (a28). Data are representative of two experiments from different donors with similar results.

FIG. 8. Activation of mTOR is required for human naive CD4 T cells to down-regulate GRAIL. A, Phospho-Akt (Ser473) and total Akt immunoblots of human naive CD4+CD45RA+ T cells after 48 h of stimulation (anti-CD3/APC), including CTLA4-Ig and anti-CD28 (a28). Data are representative of two experiments from different donors with similar results. B, Phospho-S6K1 (Thr421/Ser424), total S6K1, phospho-4E-BP1 (Thr37/46), total 4E-BP1 immunoblots of CD4 T cells as in A, after 48 h of stimulation (anti-CD3/APC), including CTLA4-Ig and anti-CD28 (a28). On total 4E-BP1 immunoblot, top arrow indicates hyperphosphorylated form and bottom arrow indicates hypophosphorylated form. Data are representative of two experiments from different donors with similar results. C, Phospho-STAT5 (Tyr^{694/699}), total STAT5, phospho-Akt (Ser⁴⁷³), total Akt, phospho-S6K1 (Thr⁴²¹/Ser⁴²⁴) total S6K1, phospho-4E-BP1 (Thr^{37/46}), and total 4E-BP1 immunoblots of CD4 T cells as in A, after 48 h of bead stimulation (anti-CD3/28), including rapamycin (RAPA). On total 4E-BP1 immunoblot, top arrow indicates hyperphosphorylated form and bottom arrow indicates hypophosphorylated form. Data are representative of three experiments from different donors with similar results. D, Proliferation assay of CD4 T cells as in A, without (−) or with (+) bead stimulation (anti-CD3/28), RAPA. Error bars indicate SD of triplicates. Measured as counts per minute. Data are representative of three experiments from different donors with similar results. E, Cyclin D3, Kip1/p27, and β-actin immunoblots of CD4 T cells as in A, after 48 h of stimulation (anti-CD3/28), including RAPA. Data are representative of three experiments from different donors with similar results. (F) GRAIL, Otu1, and β-actin immunoblots of CD4 T cells as in A, after 48 h of stimulation (anti-CD3/28), including RAPA. Data are representative of three experiments from different donors with similar results.

FIG. 9. The CD28 costimulation, IL-2 signaling, and mTOR pathway regulate Otub1 and GRAIL expression, controlling proliferation in primary naive CD4 T cells. CTLA4-Ig, anti-IL-2, and Rapamycin regulation of Otub1 and GRAIL expression controls naive CD4 T cell proliferation. A, Productive activation of naive CD4 T cells leading to proliferation comes about through TCR engagement and CD28 costimulation, IL-2 production, signaling through the IL-2R leading to phosphorylation of Akt and activation of mTOR, expression of Otub1 protein, and subsequent GRAIL degradation, allowing proliferation to occur. B, CTLA4-Ig blocks CD28 costimulation, does not allow IL-2 production, thus prevents Akt phosphorylation, mTOR is inactive, and Otub1 protein is absent, leading to the maintenance of GRAIL, inhibiting proliferation. C, Anti-IL-2 blocks IL-2R engagement, thus preventing Akt phosphorylation, mTOR is inactive, and Otub1 protein is absent, leading to the maintenance of GRAIL, inhibiting proliferation. D, Rapamycin blocks the activity of mTOR, prevents protein expression of Otubain-1, leading to the maintenance of GRAIL, inhibiting proliferation.

FIG. 10. GRAIL is expressed during quiescence, lost upon activation.

FIG. 11 Naïve cells are inhibited by a single inhibitor but effector cells are not.

FIG. 12. Naïve (A) or effector (B) T cells are stimulated with anti-CD3/28, and inhibited with the indicated drugs. Effector T cells require inhibition of two pathways to stop activation. Only the effector T cells blocked in both pathways express GRAIL (C).

FIG. 13. Schematic of activation pathways.

FIGS. 14A-14D. Combination MEK/ERK and mTOR inhibition demonstrates greatest in vivo preventive and therapeutic efficacy in a collagen-induced arthritis mouse model. (A) Percent cumulative incidence (prevalence) of arthritis disease development was measured in a prevention study in a collagen-induced arthritis mouse model. Mice were previously primed and then boosted on the indicated Day O, Seven daily injections of vehicle control (black), PD0325901 (orange), Rapamycin (green), or both drugs (blue) were administered starting from Day-1 and ending on Day 5. Number of mice in each group is indicated (n). (B) Mean visual score on Day 9 of mice as in (A) treated with vehicle control (Vehicle), PD0325901 (PD), Rapamycin (RAPA), or both drugs (PD+RAPA). Error bars indicate standard error of mean for (n) number of mice in each group as in (A). (C) Mean visual score was measured in a treatment study in a collagen-induced arthritis mouse model. Mice were primed and then boosted and subsequently monitored daily for enrolled in the treatment study upon reaching a visual disease score of 2. Daily injections of vehicle control (black), PD0325901 (orange), Rapamycin (green), or both drugs (blue) were administered. Number of mice in each group is indicated (n). (D) Mean paw thickness in millimeters (mm) was measured as in (C).

### DETAILED DESCRIPTION OF THE EMBODIMENTS

Synergistic combinations of an mTOR pathway inhibitor and a MAPK pathway inhibitor are provided, which combination of agents is effective in inactivating antigen sensitized effector CD4⁺ T cells. In some embodiments the combination of inhibitors does not comprise a tyrosine kinase inhibitor. mTOR pathway inhibitors of interest include inhibitors of mTOR, inhibitors of S6K1 and inhibitors of Akt. Inhibitors of mTOR, including rapamycin and related compounds, are of particular interest. Also of interest is CTLA4Ig, anti-IL2, and antagonist IL-2 protein. Inhibitors of the MAPK pathway of interest include inhibitors of MEK1; MEK2; ERK1; ERK2 and RSK. Of particular interest are inhibitors of the serine threonine kinase isoforms MEK1 and MEK2, which may be referred to herein as MEK.

For any of the above methods, the mTOR and MAPK inhibitors, can be administered in parallel within five days of each other, 24 hours of each other, simultaneously, or they are administered together.

Without being bound by the theory of action, it is believed that expression of the protein GRAIL, which is an active ubiquitin E3 ligase, is crucial in the induction of anergy or non-responsiveness in cells of the immune system, particularly CD4+ effector T cells, and in the regulation of cellular proliferation. Following TCR engagement in a CD4 T cell, CD28 costimulation results in the expression of IL-2 whose signaling through its receptor activates the Akt-mammalian target of rapamycin (mTOR) pathway. Activation of mTOR allows selective mRNA translation, including expression of Otubain-1 (Otub1), whose expression results in the degradation of GRAIL and allows T cell proliferation. The activation of mTOR appears to be a critical component of IL-2R signaling regulating GRAIL expression. In a naïve T cell, the expression of Otub1 can be blocked through inhibiting only the mTor pathway, for example with any one of CTLA4-Ig, anti-IL-2, or rapamycin. However, in a sensitized effector T cell, a single inhibitor is ineffective in blocking Otub1 expression. In order to block Otub1 expression in sensitized T cells it is necessary to block both the MAPK pathway, which is activated by the T cell antigen receptor, and the mTOR pathway, which is activated by IL-2 in order to block Otub1 expression.

These observations provide a mechanistic pathway sequentially linking CD28 costimulation, IL-2R signaling, and mTOR activation as important requirements for naive CD4 T cell proliferation through the regulation of Otub1 and GRAIL expression. The findings presented herein demonstrate that Otub1 is expressed and GRAIL is degraded when naive CD4 T cells are productively activated to undergo proliferation. The loss of GRAIL is mechanistically controlled through a pathway involving CD28 costimulation, IL-2 production and IL-2R signaling, and ultimately, mTOR-dependent translation of select mRNA. Interference of this pathway using CTLA4-Ig, anti-IL-2, or rapamycin prevents Otub1 protein expression and maintains GRAIL expression, which inhibits T cell proliferation.

In some embodiments of the invention, inhibitors and combinations of inhibitors are screened for efficacy by determining the presence of GRAIL protein in a T cell, in vitro or in vivo, that has been contacted with the inhibitor or combination of inhibitors, usually in combination with antigen stimulation. The presence of GRAIL under such circumstances is indicative that the cell is inactivated. Alternatively the presence of otubain1 in the cell can be assessed, where expression of Otubain1 is indicative that the cell is activated, i.e. not inhibited.

For any of the combinations described herein, the invention also features a method of determining whether an undesirable effector T cell activation in a human patient responds to a combination including an mTOR pathway inhibitor and MEK pathway inhibitor. This method includes the steps of (a) administering the combination to the human patient or to ex vivo T cells obtained from the human patient; and (b) monitoring the T cells to determine whether the undesirable effector T cell activation responds to the combination, e.g. by determining the expression of either Otubain1 or GRAIL in T cells in the presence of antigenic stimulation, e.g. anti-CD3. This method can be performed, for example, to determine whether the combination has enhanced efficacy in comparison to monotherapy using any one of the inhibitors in the combination. This method can also be used to determine which regimens are effective for treating the undesirable effector T cell activation (e.g., variables include the amount of each inhibitor in the combination, routes of administration for each inhibitor, and/or the intervals between administrations).

Administration of the mTOR and MEK inhibitors can be achieved by a variety of routes, such as by parenteral routes (e.g., intravenous, intraarterial, intramuscular subcutaneous injection), topical, inhalation (e.g., intrabronchial, intranasal or oral inhalation or intranasal drops), oral, rectal, or other routes.

The present invention also features a pharmaceutical composition including an effective amount of a rapamycin macrolide and a MEK inhibitor, which combination usually lacks a tyrosine kinase inhibitor, together with a pharmaceutically acceptable carrier or diluent.

### DEFINITIONS

T cell inactivation. As used herein, the term T cell inactivation refers to a non-responsive phenotype in a CD4⁺ T cell, where the cell does not proliferate in response to normal antigenic stimulation. Unless specifically noted, the CD4⁺ T cell is a previously stimulated effector cell, i.e. a cell other than a naïve T cell.

As is known in the art, a naive T cell or Th0 cell is a T cell that has differentiated in bone marrow, and successfully undergone the positive and negative processes of central selection in the thymus. A naive T cell is considered mature, but is distinguished from activated T cells or memory T cells, as it is has not yet encountered cognate antigen in the periphery. Naive T cells may be characterized by the surface expression of L-selectin (CD62L); the absence of the activation markers CD25, CD44 or CD69; and the absence of memory markers, such as the edited CD45 isoforms. In the naive state, T cells are thought to be quiescent and non-dividing, requiring the common-gamma chain cytokines IL-7 and IL-15 for homeostatic survival.

Recognition by a naive T cell clone of its cognate antigen results in the initiation of an acquired immune response. In the ensuing response, the T cell acquires an activated phenotype (CD25⁺, CD44⁺, CD62^{low}, CD69⁺), and may further differentiate into a memory T cell.

mTOR pathway. In T cells, binding of a ligand to the IL-2 receptor activates a signaling pathway mediated through a cascade of proteins of which akt, mTOR and S6K-1 are members. Activation of the signaling cascade results in expression of Otubain1, which degrades GRAIL and allows release of the T cell from the non-responsive state. The mammalian target of rapamycin (mTOR) is one of a family of proteins involved in cell cycle progression, DNA recombination, and DNA damage detection. The cDNA for mTOR (which may also be referred to as Homo sapiens FK506 binding protein 12-rapamycin associated protein 1 (FRAP1) encodes a predicted 2,549-amino acid protein with a molecular mass of approximately 300 kD. The genetic sequences of the mRNA and protein may be accessed at Genbank, NM_{—}004958.3. The N-terminal half of the protein contains 20 tandem HEAT repeats, which are implicated in protein-protein interactions. Each HEAT repeat consists of 2 alpha helices of about 40 amino acids. The C-terminal half contains a large FRAP-ATM-TRRAP (FAT) domain, followed by the FKB12- and rapamycin-binding domain, a serine/threonine kinase catalytic domain, a negative regulatory domain, and a C-terminal FAT (FATC) domain necessary for MTOR activity. Inhibitors of the mTOR pathway include, without limitation, rapamycin and related compounds, CTLA4Ig, antagonistic IL-2, and anti-IL2. Alternative inhibitors include antibodies, antisense and RNAi inhibitors of mTOR or proteins in the mTOR signaling pathway.

Antagonist biologic IL-2 protein is of interest as an mTOR inhibitor. Wildtype IL-2 normally binds the heterotrimeric IL-2R complex composed of IL-2Ra, IL-2Rb, and common gamma chain. Antagonist IL-2 comprises mutations introduced at the amino acid residues that contact the common gamma chain, rendering null the signaling capacity through the Akt-mTOR pathway. In doing so, the antagonist IL-2 binds to IL-2Ra and IL-2Rb but absent common gamma chain binding will not only lack signaling but block any endogenous wildtype IL-2 from signaling through the IL-2R complex. Additional modifications may be made to the antagonist IL-2 at the amino acid residues that contact the IL-2Rb to increase its affinity to better compete against endogenous wildtype IL-2 in a dominant-negative fashion. The antagonist IL-2 can be used in the combinatorial therapy of the present invention. The use of the antagonist IL-2 rather than two small molecules in combination therapy is likely to result in less toxicity due to greater specificity of the antagonist IL-2 against immune cell mTOR signaling, particularly T cell mTOR signaling.

The macrolide fungicide rapamycin is a natural product that binds intracellularly to the immuunophilin FK506 binding protein 12 (FKBP12), and the resultant complex inhibits the serine protein kinase activity of mammalian target of rapamycin (mTOR). The inhibition of mTOR, in turn, blocks signals to at least two separate downstream pathways which control the translation of specific mRNAs required for cell proliferation, including Otubain1. Related mTOR inhibitors include, without limitation, any of the rapamycin macrolides, e.g. rapamycin, CCI-779, Everolimus, and ABT-578.

Wherever the present application refers to “rapamycin macrolide”, in addition to naturally occurring forms of rapamycin, the invention further includes rapamycin analogs and derivatives. Many such analogs and derivatives are known in the art. Examples include those compounds described in U.S. Pat. Nos. 6,329,386; 6,200,985; 6,117,863; 6,015,815; 6,015,809; 6,004,973; 5,985,890; 5,955,457; 5,922,730; 5,912,253; 5,780,462; 5,665,772; 5,637,590; 5,567,709; 5,563,145; 5,559,122; 5,559,120; 5,559,119; 5,559,112; 5,550,133; 5,541,192; 5,541,191; 5,532,355; 5,530,121; 5,530,007; 5,525,610; 5,521,194; 5,519,031; 5,516,780; 5,508,399; 5,508,290; 5,508,286; 5,508,285; 5,504,291; 5,504,204; 5,491,231; 5,489,680; 5,489,595; 5,488,054; 5,486,524; 5,486,523; 5,486,522; 5,484,791; 5,484,790; 5,480,989; 5,480,988; 5,463,048; 5,446,048; 5,434,260; 5,411,967; 5,391,730; 5,389,639; 5,385,910; 5,385,909; 5,385,908; 5,378,836; 5,378,696; 5,373,014; 5,362,718; 5,358,944; 5,346,893; 5,344,833; 5,302,584; 5,262,424; 5,262,423; 5,260,300; 5,260,299; 5,233,036; 5,221,740; 5,221,670; 5,202,332; 5,194,447; 5,177,203; 5,169,851; 5,164,399; 5,162,333; 5,151,413; 5,138,051; 5,130,307; 5,120,842; 5,120,727; 5,120,726; 5,120,725; 5,118,678; 5,118,677; 5,100,883; 5,023,264; 5,023,263; and 5,023,262; all of which are incorporated herein by reference.

Desirable rapamycin macrolides for use in the present methods include rapamycin, CCI-779, Everolimus (also known as RAD001), and ABT-578. CCI-779 is an ester of rapamycin (42-ester with 3-hydroxy-2-hydroxymethyl-2-methylpropionic acid), disclosed in U.S. Pat. No. 5,362,718. Everolimus is an alkylated rapamycin (40-O-(2-hydroxyethyl)-rapamycin, disclosed in U.S. Pat. No. 5,665,772.

MAPK (MEK) pathway. The MAPK/ERK pathway is a signal transduction pathway that couples intracellular responses to the binding of growth factors to cell surface receptors. This pathway is complex and includes many protein components. In many cell types, activation of this pathway promotes cell division. Activated Ras activates the protein kinase activity of RAF kinase. RAF kinase phosphorylates and activates MEK (mitogen/extracellular-signal-regulated kinase kinase). MEK phosphorylates and activates a mitogen-activated protein kinase (MAPK). RAF, MEK and MAPK are all serine/threonine-selective protein kinases. One effect of MAPK activation is to alter the translation of mRNA to proteins. MAPK phosphorylates 40S ribosomal protein S6 kinase (RSK). This activates RSK which in turn phosphorylates ribosomal protein S6.

MEK is a dual-specificity kinase that phosphorylates the tyrosine and threonine residues on MAPK or extracellular signal-regulated kinase (ERK) required for activation. Two related genes encode MEK1 and MEK2. MEKs are substrates for several protein kinases including the Rafs (c-, A- and B-), Mos, Tpl-2, and MEKK1. MEKs are phosphorylated by these kinases at two serine residues.

ERK can phosphorylate several of the members of the ETS family of transcription factors, explaining its apparent ability to activate transcription of certain genes. ERK can also activate a variety of protein kinases via phosphorylation. For example, p90 RSK is a serine-threonine kinase that has a role in protein translation and has been shown to be a substrate for the ERKs.

The genetic sequence of human ERK proteins can be accessed at Genbank, NM_{—}001109891; NM_{—}002746; NM_{—}001040056.1, herein incorporated by reference. The genetic sequence of human MEK can be accessed at Genbank, NM_{—}002755.3. The RSK (ribosomal S6 kinase) family comprises growth factor-regulated serine/threonine kinases, known also as p90(rsk), sequences may be accessed at Genbank, NM_{—}002953.3; and NM_{—}001006665.1. All such sequences are herein incorporated by reference.

MEK Inhibitors. Any MEK inhibitor can be used in the methods of the present invention. MEK inhibitors can be identified using known MEK inhibition assays. For example, the assays described in U.S. Pat. No. 5,525,625 or in WO 02/06213 A1, can be used to identify MEK inhibitors. Examples of MEK inhibitors include those compounds described in U.S. Pat. Nos. 6,545,030, 6,506,798, 6,492,363, 6,469,004, 6,455,582, 6,440,966, 6,310,060, 6,214,851, and 5,525,625, and U.S. Publication Nos. US 2003/0092748 A1, US 2003/0078428 A1, US 2003/0045521 A1, US 2003/0004193 A1, and US 2002/0022647 A1. Inhibitors of interest include, without limitation, PD184352/CI-1044 (Pfizer); PD198306 (Pfizer); PD98059 (Pfizer); U0126 (Promega); Ro092210 (Roche); and L783277 (Merck). Inhibitors may also include antisense and siRNA molecules that specifically target a protein in the MAPK/MEK signaling pathway.

Inhibitors of other proteins in the signaling pathway also find use, e.g. inhibitors of RSK, e.g. SL0101; inhibitors of ERK; etc.

Compounds useful in the present invention include those described herein in any of their pharmaceutically acceptable forms, including isomers such as diastereomers and enantiomers, salts, and solvates thereof, as well as racemic mixtures of the compounds described herein.

The terms “administration” and “administering” refer to a method of giving a dosage of a pharmaceutical composition to a patient, where the method is, e.g., topical, oral, intravenous, intraperitoneal, or intramuscular. The preferred method of administration can vary depending on various factors, e.g., the components of the pharmaceutical composition, site of the potential or actual disease and severity of disease.

By administration “in parallel” is meant that the mTOR inhibitor and the MEK inhibitor are formulated separately and administered separately.

By administered “together” is meant that the mTOR inhibitor and the MEK inhibitor are formulated together in a single pharmaceutical composition and administered together.

By “effective amount” is meant the amount of a compound required to reduce effector CD4+T cell activity. The effective amount of mTOR inhibitor and MEK inhibitor used to practice the present invention for the treatment of an undesirable effector T cell activation varies depending upon the manner of administration, the age, body weight, and general health of the subject. Ultimately, the attending physician, will decide the appropriate amount and dosage regimen. Such amount is referred to as an “effective” amount.

As used herein, “individual” or “patient” includes humans, cattle, pigs, sheep, horses, dogs, and cats, and also includes other vertebrates, most preferably, mammalian species.

By “rapamycin macrolide” is meant naturally occurring forms of rapamycin in addition to rapamycin analogs and derivatives which target and inhibit mTOR.

By “small molecule” inhibitor is meant a molecule of less than about 3,000 daltons having antagonist activity against a protein in the MAPK or mTOR signaling pathway.

By “antisense” or “antisense oligomer” is meant any oligonucleotide or oligonucleoside that acts to inhibit the expression or function of a protein.

By “RNAi inhibitor” is meant any double stranded RNA that acts to inhibit the expression or function of a protein (for an example of RNAi technology, see Zamore et al., Cell 101:25-33 (2000)).

GRAIL. As used herein, the term “GRAIL” refers to the polypeptide and polynucleotides disclosed in co-pending U.S. patent application U.S. Ser. No. 09/854,300, including variants, homologs and polymorphisms thereof. The GRAIL protein is an E3 ligase, which has the enzymatic activity of ligating ubiquitin to itself, and to its substrates. The presence of high levels of GRAIL protein is indicative of cells that are anergic, or have a low capacity for proliferation.

Otubain Isoforms. The Otubain gene is differentially spliced to give rise to a number of distinct isoforms, which act as regulators of GRAIL, and therefore of anergy and cellular proliferation, which may be referred to human SOG and DOG. SOG is encoded by an mRNA of about 950 bp, giving rise to a protein of about 35 KDa. The DOG transcript is 815 bp, encoding a 31 KDa protein. The two isoforms differ by an additional 210 by of coding sequence in the central region of the mRNA. DOG is a ubiquitin specific protease with specificity toward isolated branched polyubiquitin chains, and is widely expressed, whereas SOG is preferentially expressed in secondary lymphoid tissues. The shorter isoform cDNA and the larger isoform to a 35 KDa protein. Both DOG and SOG have identical c-terminal 140 amino acids, including the GRAIL binding domain. However, SOG lacks two of the three amino acids (Asp and Cys) that compose the signature OTU cysteine proteinase catalytic core of the original OTUBAIN-1, and retains only the C-terminal flanking region of the OTU domain, and therefore lacks the catalytic activity of DOG.

USP8. As used herein, USP8 is a deubiquitinating enzyme. The human sequence may be accessed at Genbank, accession no. D29956. The sequence of USP8 displays the typical hallmarks of the UBP family of de-ubiquitinating enzymes, including the so called histidine and cysteine boxes. There is evidence that it is a phosphoprotein. USP8 can hydrolyze ubiquitin-isopeptide bonds, and linear ubiquitin chains. The protein product appears as a doublet of approximately 130 KDa. Expression is reduced in growth arrested or GO cells, and increased in proliferating cells.

### Therapeutic/Prophylactic Treatment Methods

A combined synergistic formulation of two compounds (a) an inhibitor of the MEK signaling pathway in T cells and (b) an inhibitor of the mTOR signaling pathway in T cells is contacted with a composition of effector CD4⁺ T cells. Numerous agents are useful in modulating this activity, including agents that directly modulate expression, e.g. antisense specific for the targeted proteins; and agents that act on the protein, e.g. specific antibodies and analogs thereof, small organic molecules that block catalytic activity, etc. Agents may be administered to patients suffering from autoimmune or immune tolerance disorders, hyperproliferative conditions, etc. The formulation may be free of tyrosine kinase inhibitors, e.g. imatinib, and the like. In some embodiments the T cells are present in a mixed population, which may comprise naïve T cells as well as other cells involved in immune reactions. In some embodiments the T cells are present in vitro. In other embodiments the T cells are present in vivo. Where the T cells are present in vivo, the host individual may be suffering from undesirable T cell activity, particularly in the context of graft rejection, or autoimmune disease.

As used herein, the term “treating” is used to refer to both prevention of disease, and treatment of pre-existing conditions. The prevention of proliferation is accomplished by administration of the compounds prior to development of overt disease, e.g., to prevent the development of autoimmune disease; diminish graft rejection or autoimmune disease, etc. Alternatively the compounds are used to treat ongoing disease, by stabilizing or improving the clinical symptoms of the patient. The subject therapy may be administered during the presymptomatic or preclinical stage of the disease, and in some cases during the symptomatic stage of the disease. Early treatment is preferable, in order to prevent the loss of function associated with inflammatory tissue damage. The presymptomatic, or preclinical stage will be defined as that period not later than when there is T cell involvement at the site of disease, e.g. islets of Langerhans, synovial tissue, thyroid gland, etc., but the loss of function is not yet severe enough to produce the clinical symptoms indicative of overt disease. T cell involvement may be evidenced by the presence of elevated numbers of T cells at the site of disease, the presence of T cells specific for autoantigens, the release of performs and granzymes at the site of disease, response to immunosuppressive therapy, etc.

The susceptibility of a particular cell or tissue to treatment with the subject compounds may be determined by in vitro testing. Typically a culture of the cell is combined with a subject compound at varying concentrations for a period of time sufficient to allow the active agents to induce anergy, usually between about one hour and one week. For in vitro testing, cultured cells from a biopsy sample may be used. The cycling cells left after treatment are then counted.

The dose will vary depending on the specific compound utilized, specific disorder, patient status, etc. For treatment of tumors, typically a therapeutic dose will be sufficient to substantially decrease the proliferation of the undesirable cell population in the targeted tissue, while maintaining patient viability. For treatment of autoimmune disease, therapeutic effects may be measured by a decrease in immune responsiveness against the target antigen; or decrease in patient symptoms, e.g. the presence of antinuclear antibodies in SLE; and the like.

The proliferation of immune cells is associated with a number of autoimmune and lymphoproliferative disorders. Diseases of interest include multiple sclerosis, rheumatoid arthritis and insulin dependent diabetes mellitus. Evidence suggests that abnormalities in apoptosis play a part in the pathogenesis of systemic lupus erythematosus (SLE). Symptoms of allergies to environmental and food agents, as well as inflammatory bowel disease, may also be alleviated by the treatment of the invention.

Specific conditions of interest include systemic lupus erythematosus (SLE) is an autoimmune disease characterized by polyclonal B cell activation, which results in a variety of anti-protein and non-protein autoantibodies (see Kotzin et al. (1996) Cell 85:303-306 for a review of the disease). These autoantibodies form immune complexes that deposit in multiple organ systems, causing tissue damage. SLE is a difficult disease to study, having a variable disease course characterized by exacerbations and remissions. For example, some patients may demonstrate predominantly skin rash and joint pain, show spontaneous remissions, and require little medication. The other end of the spectrum includes patients who demonstrate severe and progressive kidney involvement (glomerulonephritis) that requires therapy with high doses of steroids and cytotoxic drugs such as cyclophosphamide.

It appears that multiple factors contribute to the development of SLE. Several genetic loci may contribute to susceptibility, including the histocompatibility antigens HLA-DR2 and HLA-DR3. The polygenic nature of this genetic predisposition, as well as the contribution of environmental factors, is suggested by a moderate concordance rate for identical twins, of between 25 and 60%.

Disease manifestations result from recurrent vascular injury due to immune complex deposition, leukothrombosis, or thrombosis. Additionally, cytotoxic antibodies can mediate autoimmune hemolytic anemia and thrombocytopenia, while antibodies to specific cellular antigens can disrupt cellular function. An example of the latter is the association between anti-neuronal antibodies and neuropsychiatric SLE.

Degenerative joint diseases may be inflammatory, as with seronegative spondylarthropathies, e.g. ankylosing spondylitis and reactive arthritis; rheumatoid arthritis; gout; and systemic lupus erythematosus. The degenerative joint diseases have a common feature, in that the cartilage of the joint is eroded, eventually exposing the bone surface. Destruction of cartilage begins with the degradation of proteoglycan, mediated by enzymes such as stromelysin and collagenase, resulting in the loss of the ability to resist compressive stress. Alterations in the expression of adhesion molecules, such as CD44 (Swissprot P22511), ICAM-1 (Swissprot P05362), and extracellular matrix protein, such as fibronectin and tenascin, follow. Eventually fibrous collagens are attacked by metalloproteases, and when the collagenous microskeleton is lost, repair by regeneration is impossible. There is significant immunological activity within the synovium during the course of inflammatory arthritis. While treatment during early stages is desirable, the adverse symptoms of the disease may be at least partially alleviated by treatment during later stages. Clinical indices for the severity of arthritis include pain, swelling, fatigue and morning stiffness, and may be quantitatively monitored by Pannus criteria. Disease progression in animal models may be followed by measurement of affected joint inflammation. Therapy for inflammatory arthritis may combine the subject treatment with conventional NSAID treatment.

A quantitative increase in myelin-autoreactive T cells with the capacity to secrete IFN-gamma is associated with the pathogenesis of MS and EAE, suggesting that autoimmune inducer/helper T lymphocytes in the peripheral blood of MS patients may initiate and/or regulate the demyelination process in patients with MS. The overt disease is associated with muscle weakness, loss of abdominal reflexes, visual defects and paresthesias. During the presymptomatic period there is infiltration of leukocytes into the cerebrospinal fluid, inflammation and demyelination. Family histories and the presence of the HLA haplotype DRB1*1501, DQA1*0102, DQB*0602 are indicative of a susceptibility to the disease. Markers that may be monitored for disease progression are the presence of antibodies in the cerebrospinal fluid, “evoked potentials” seen by electroencephalography in the visual cortex and brainstem, and the presence of spinal cord defects by MRI or computerized tomography. Treatment during the early stages of the disease will slow down or arrest the further loss of neural function.

Human IDDM is a cell-mediated autoimmune disorder leading to destruction of insulin-secreting β cells and overt hyperglycemia. T lymphocytes invade the islets of Langerhans, and specifically destroy insulin-producing.beta.-cells. The depletion of β cells results in an inability to regulate levels of glucose in the blood. Overt diabetes occurs when the level of glucose in the blood rises above a specific level, usually about 250 mg/dl.

In humans a long presymptomatic period precedes the onset of diabetes. During this period there is a gradual loss of pancreatic.beta. cell function. The disease progression may be monitored in individuals diagnosed by family history and genetic analysis as being susceptible. The most important genetic effect is seen with genes of the major histocompatibility locus (IDDM1), although other loci, including the insulin gene region (IDDM2) also show linkage to the disease (see Davies et al, supra and Kennedy et al. (1995) Nature Genetics 9:293-298). Markers that may be evaluated during the presymptomatic stage are the presence of insulitis in the pancreas, the level and frequency of islet cell antibodies, islet cell surface antibodies, aberrant expression of Class II MHC molecules on pancreatic β cells, glucose concentration in the blood, and the plasma concentration of insulin. An increase in the number of T lymphocytes in the pancreas, islet cell antibodies and blood glucose is indicative of the disease, as is a decrease in insulin concentration. After the onset of overt diabetes, patients with residual.beta. cell function, evidenced by the plasma persistence of insulin C-peptide, may also benefit from the subject treatment, to prevent further loss of function.

The response of the host immune system to a graft, or of a graft towards the host (GVHD) is reduced by treatment with the subject synergistic combination of inhibitors. Grafts include the transplantation of cells, tissues and organs, such as the transfusion of blood or blood components, the grafting of bone, skin, bone marrow, etc., and the transplantation of tissues of the eye, pancreas, liver, kidney, heart, brain, bowel, lung, etc. Of interest are transplantation of hematopoietic cells, e.g. bone marrow, mobilized hematopoietic stem cells in peripheral blood, etc., transplantation of kidneys and transplantation of hearts. As used herein, a graft recipient is an individual to whom tissue or cells from another individual (donor), commonly of the same species, has been transferred, particularly where one or more of the Class I MHC antigens are different in the donor as compared to the recipient. However, in some instances xenogeneic, e.g. pig, baboon, etc., tissue, cells or organs will be involved. The graft recipient and donor are generally mammals, preferably human.

### PHARMACEUTICAL COMPOSITIONS

Inhibitors and combinations of inhibitors can serve as the active ingredient in pharmaceutical compositions formulated for the treatment of disorders associated with activation of effector T cells, including lack of immune tolerance, autoimmune disease, etc. The compositions can also include various other agents to enhance delivery and efficacy. For instance, compositions can include agents capable of increasing the bioavailability of the compound. The compositions can also include various agents to enhance delivery and stability of the active ingredients.

Thus, for example, the compositions can also include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with little or no toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (La, they can be “nebulized”) to be administered via inhalation. Aerosol formulations can be placed into pressurized acceptable propellants, such as dichlorodifluoromethane, propane, nitrogen.

Suitable formulations for rectal administration include, for example, suppositories, which consist of the packaged active ingredient with a suppository base. Suitable suppository bases include natural or synthetic triglycerides or paraffin hydrocarbons. In addition, it is also possible to use gelatin rectal capsules, which consist of a combination of the packaged active ingredient with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### Screening Methods

For any of the combinations described herein, the invention also features a method of determining whether an effector T cell population responds to the combination of inhibitors, including the use of the method in optimizing dose and combinations of agent. In such methods, a combination of inhibitors, where one inhibitor acts of the mTOR pathway and the other inhibitor acts on the MEK pathway in an effector T cell, is brought into contact with a population of effector T cells. The population may be isolated, present in an in vitro culture in combination with other cells, or present in an animal, where the animal may, for example, provide a model for an immune dysfunction. Typically multiple doses are utilized, and multiple ratios, for example a 1:1 ratio of the first inhibitor and the second inhibitor, a 1:2 ratio; 2:1, 1:5; 5:1, 1:10; 10:1, 1:50; 50:1, and the like, where the ratio may be provided as either weight/weight; or units of specific activity to units of specific activity. The dose may also be varied, where the initial dose may be the dose utilized in monotherapy, such as the normal dose for Sirolimus, e.g. a single loading dose of 15 mg and initial maintenance dose of 5 mg/day for a human patient. The dose may then be modified in serial dilutions, e.g. a maintenance dose of 5 mg; 2.5 mg; 1 mg; 0.5 mg; etc. of Sirolimus, in combination with a suitable dose of a MEK pathway inhibitor.

The effectiveness and optimization of the therapy can be determined by T cell proliferation, using standard methods; or by determining the expression of GRAIL, Otubain1; and related proteins in the targeted T cells. This method can be performed, for example, to determine whether the combination has enhanced efficacy in comparison to monotherapy using any one of the inhibitors in the combination. This method can also be used to determine which regimens are effective for treating the undesirable effector T cell activation (e.g., variables include the amount of each inhibitor in the combination, routes of administration for each inhibitor, and/or the intervals between administrations).

### EXPERIMENTAL

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

Abbreviations used include: GRAIL, gene related to anergy in lymphocyte; mTOR, mammalian target of rapamycin; Otub1, Otubain-1; pOVA, peptide from ovalbumin.

### Example 1

### Naive CD4 T Cell Proliferation is Controlled by Mammalian Target of Rapamycin Regulation of GRAIL Expression

In this study, we demonstrate that the E3 ubiquitin ligase gene related to anergy in lymphocytes (GRAIL) is expressed in quiescent naive mouse and human CD4 T cells and has a functional role in inhibiting naive T cell proliferation. Following TCR engagement, CD28 costimulation results in the expression of IL-2 whose signaling through its receptor activates the Akt-mammalian target of rapamycin (mTOR) pathway. Activation of mTOR allows selective mRNA translation, including the epistatic regulator of GRAIL, Otubain-1 (Otub1), whose expression results in the degradation of GRAIL and allows T cell proliferation. The activation of mTOR appears to be the critical component of IL-2R signaling regulating GRAIL expression. CTLA4-Ig treatment blocks CD28 costimulation and resultant IL-2 expression, whereas rapamycin and anti-IL-2 treatment block mTOR activation downstream of IL-2R signaling. Thus, all three of these biotherapeutics inhibit mTOR-dependent translation of mRNA transcripts, resulting in blockade of Otub1 expression, maintenance of GRAIL, and inhibition of CD4 T cell proliferation. These observations provide a mechanistic pathway sequentially linking CD28 costimulation, IL-2R signaling, and mTOR activation as important requirements for naive CD4 T cell proliferation through the regulation of Otub1 and GRAIL expression. Our findings also extend the role of GRAIL beyond anergy induction and maintenance, suggesting that endogenous GRAIL regulates general cell cycle and proliferation of primary naive CD4 T cells.

Although a role for GRAIL in regulating CD4 T cell proliferation has been demonstrated in clones and in transgenic expression systems, little is known about the expression, regulation, or function of endogenous GRAIL or Otub1 in naive CD4 T cells. In this study, we investigated how the expression of GRAIL and Otub1 is regulated during mouse and human naive CD4 T cell activation. Our findings demonstrate that Otub1 is expressed and GRAIL is degraded when naive CD4 T cells are productively activated to undergo proliferation. The loss of GRAIL is mechanistically controlled through a pathway involving CD28 costimulation, IL-2 production and IL-2R signaling, and ultimately, mTOR-dependent translation of select mRNA. Interference of this pathway using CTLA4-Ig, anti-IL-2, or rapamycin prevents Otub1 protein expression and maintains GRAIL expression, which inhibits T cell proliferation.

### Materials and Methods

Mice. BALB/c, DO11, NOD, and NOD.B10 female mice were purchased from The Jackson Laboratory. DO11 CD28−/− female mice were a gift from Drs. A. Abbas and L. Barron (University of California, San Francisco, Calif.). All procedures involving mice were conducted in accordance with Institutional Animal Care and Use Committee policies as set forth by Stanford University's Administrative Panel on Laboratory Animal Care, as accredited by the Association for Assessment and Accreditation of Laboratory Animal Care International.

Isolation and stimulation of mouse CD4 T cells. Spleen and lymph nodes were harvested from naive mice and homogenized through a strainer. RBC were lysed from the suspension using red blood cell lysing buffer (Sigma-Aldrich). Lymphocytes were isolated by density centrifugation using Lympholyte-M (Cedarlane Laboratories). CD4+ T cells were sorted via negative selection using an AutoMACS sorter (Miltenyi Biotec). BALB/c CD4+ T cells (5×103) were stimulated in 96-well U-bottom plates with equal numbers of polystyrene latex beads (Interfacial Dynamics) coated with 1.0 μg/ml anti-CD3 (145-2C11; eBioscience) and 0.5 μg/ml anti-CD28 (37.51; eBioscience). For DO11 T cells, 5×103 DO11 CD4+ T cells were stimulated in 96-well U-bottom plates with 104 APC and 50 ng/ml peptide OVA323-339 (pOVA). Rapamycin (Sigma-Aldrich) was used at a concentration of 100 nM. CTLA4-Ig (Abatacept; Bristol-Myers Squibb) was used at a concentration of 10 μg/ml. Anti-IL-2 Ab (JES6-1A12; eBioscience) was used at a concentration of 10 μg/ml. Cells were cultured in RPMI 1640 medium (Life Technologies) supplemented with 10% heat-inactivated FCS (Mediatech), 100 nM sodium pyruvate (Life Technologies), 2 mM L-glutamine (Life Technologies), 100 nM nonessential amino acids (Life Technologies), 100 U/ml penicillin/streptomycin (Life Technologies), and 5 nM 2-ME (Sigma-Aldrich).

Isolation and stimulation of human naive CD4⁺ CD45RA⁺ T cells. Human peripheral blood mononucleated cells from buffy coats of different donors were obtained from the Stanford Blood Center under Stanford University Institutional Review Board approval. Buffy coats were separated into leukocytes using Ficoll-Paque Plus (GE Health Sciences). T cells were prepared using a RosetteSep Human CD4 T Cell Enrichment (Stem Cell Technologies) followed by a Naive CD4⁺ T cell Isolation Kit along using LS MACS columns (Miltenyi Biotec). Negatively selected CD4+CD45RA+CD45RO-CD25-T cells were isolated at 95-99% purity as confirmed by flow cytometry using anti-CD4-FITC (OKT4; eBioscience) and anti-CD45RA-PE (HI100; eBioscience) Ab. CD4⁺ CD45RA⁺ T cells (5×10³) were stimulated in 96-well U-bottom plates with equal numbers of Dynabeads CD3/28 T Cell Expander (Invitrogen) or plate-bound anti-CD3 at 1.0 μg/ml with mitomycin C (Sigma-Aldrich) inactivated APC (anti-CD3/APC). Rapamycin (Sigma-Aldrich) was used at a concentration of 100 nM, and CTLA4-Ig (Abatacept, Bristol-Myers Squibb) was used at a concentration of 10 μg/ml. Agonist anti-CD28 Ab was used at 1.0 μg/ml (CD28.2; eBioscience). Anti-IL-2 Ab (5334; eBioscience) was used at a concentration of 10 μg/ml. Recombinant human IL-2 (PeproTech) was used at a concentration of 10 ng/ml. Cells were cultured in X-Vivo 15 medium (Lonza) supplemented with 10% heat-inactivated FCS (Mediatech), 2 mM L-glutamine (Life Technologies), 100 U/ml penicillin/streptomycin (Life Technologies), and 5 nM 2-ME (Sigma-Aldrich).

Proliferation and cell division assays. Cells cultured in 96-well U-bottom plate wells were pulsed with 1 μCi of methyl-[³H]thymidine (Amersham Biosciences) for 6 h during the last 72 h of stimulation and harvested onto filters (Wallac). Filters were wetted with Betaplate Scintillation fluid (PerkinElmer) and counts per minute read on a 1205 Betaplate Liquid Scintillation Counter (Wallac). For CFSE experiments, cells were labeled with 1 μM CFDA-SE (Sigma-Aldrich) in serum free RPMI 1640 medium for 10 min and washed twice before culturing. CFSE-labeled cells were assayed after 72 h of culture.

Immunoblots. Whole-cell lysates were made using lysis buffer consisting of 0.5% Nonidet P-40, 100 mM sodium chloride, 0.5 mM EDTA, 20 mM Tris (pH 7.6-8.0), with protease inhibitor mixture (Pierce) and phosphatase inhibitor mixture (Pierce). Protein samples were loaded on 4-15% Tris-HCl gels (Bio-Rad) and separated by SDS-PAGE. Protein was transferred from gel to Immobolin-P polyvinylidene difluoride membrane (Millipore) using Trans-Blot SD Semidry Transfer Apparatus (Bio-Rad) following the manufacturer's instructions. StartingBlock (Tris-buffered saline with 0.05% Tween 20) (Pierce) was used to block membranes and was also used during primary and secondary Ab staining. Secondary Abs were all HRP conjugated (Zymed Laboratories). ECL Plus Western Blotting Reagents (GE Healthcare) were used for chemiluminescent detection of protein. Chemiluminescence signal was exposed onto Amersham Hyperfilm ECL (GE Healthcare). Membranes were stripped using Restore Western Blot Stripping Buffer (Pierce). Densitometry was performed using ImageJ software (National Institutes of Health). Primary Abs used were anti-phospho-4EBP1 (Thr37/46) (236B4; Cell Signaling Technology), anti-4E-BP1 (53H11; Cell Signaling Technology), anti-β-actin (ab8226; Abcam), anti-phospho-Akt (Ser473) (44-623G; Invitrogen), anti-Akt (9272; Cell Signaling Technology), anti-cyclin D3 (1/cyclin D3; BD Biosciences), anti-GAPDH (ab9485; Abcam), anti-GRAIL (affinity-purified rabbit polyclonal) or anti-GRAIL (H11-744; BD Biosciences), anti-Kip1/p27 (57; BD Biosciences), anti-Otub1 (mouse monoclonal, a gift from Berlex Biosciences), anti-phospho-S6K1 (Thr421/Ser424) (9204; Cell Signaling Technology), anti-S6K1 (9202; Cell Signaling Technology), antiphospho-STAT5 (Tyr694/699) (8-5-2, Upstate Biotechnology), and anti-STAT5 (9363; Cell Signaling Technology).

Flow cytometry. Samples were stained and washed in PBS with 0.5% BSA and 0.02% sodium azide. Anti-CD25-PE (PC61; BD Biosciences) staining was used at (1/100) dilution on ice, in the dark, for 15 min. Samples were acquired using an LSR flow cytometer (BD Biosciences).

ELISA. Supernatant was collected 24 h after stimulation. Anti-IL-2 capture Ab (JES6-1A12, BD Biosciences) and biotinylated detection Ab (JES6-5H4; BD Biosciences) were used according to the manufacturer's instructions. Detection using ExtrAvidin Peroxidase conjugate (Sigma-Aldrich) and 3,3′,5,5′-Tetramethylbenzidine Liquid Substrate System (Sigma-Aldrich) were used according to the manufacturer's instructions.

Microarray analysis. Microarray data of NOD vs NOD.B10 pancreatic lymph node mRNA expression is publicly available and was analyzed using Matrix2png software.

Real-time quantitative PCR. RNA was collected from samples using RNeasy Kit (Qiagen). RNA was reverse transcribed into cDNA using Omniscript RT Kit (Qiagen), with DNase Set (Qiagen). Real-time quantitative PCR was conducted using Brilliant qPCR SYBR Green Mastermix (Stratagene) according to the manufacturer's instructions, and cDNA samples were run on an Mx4000 thermocycler (Stratagene). Primers used for mouse GRAIL: (F) 5′-GCGCAGTCAGCAAATGAA-3′, (R) 5′-TGTCAACATGGGGAACAACA-3′; mouse IL-2: (F) 5′-CCTGAGCAGGATGGAGAATTACA-3′, (R) 5′-TCCAGAACATGCCGCAGAG-3′; mouse Otub1: (F) 5′-CGACTCCGAAGGTGTTAACTGT-3′, (R) 5′-GAGGTCCTTGATCTTCTGTTGG-3′; and mouse β-actin: (F) 5′-CAGGCATTGCTGACAGGATGCA-3′, (R) 5′-GGCCAGGATGGAGCCACCGATC-3′.

Retroviral transduction. Retroviral transduction was performed as described previously (Lin et al. (2005) J. Immunol. 174: 5950-5958). Murine GRAIL (Rnf128) cDNA was cloned into the MSCV-IRES-GFP vector, denoted as MSCV-GRAIL-IRES-GFP (GRAIL-expressing). MSCVGRAIL-IRES-GFP and MSCV-IRES-GFP (vector control) retroviral vectors were used to generate retrovirus for CD4 T cell transduction experiments. The MSCV-IRES-GFP retroviral vector was a gift from Drs. K. Murphy and T. Murphy (Washington University, St. Louis, Mo.).

### Results

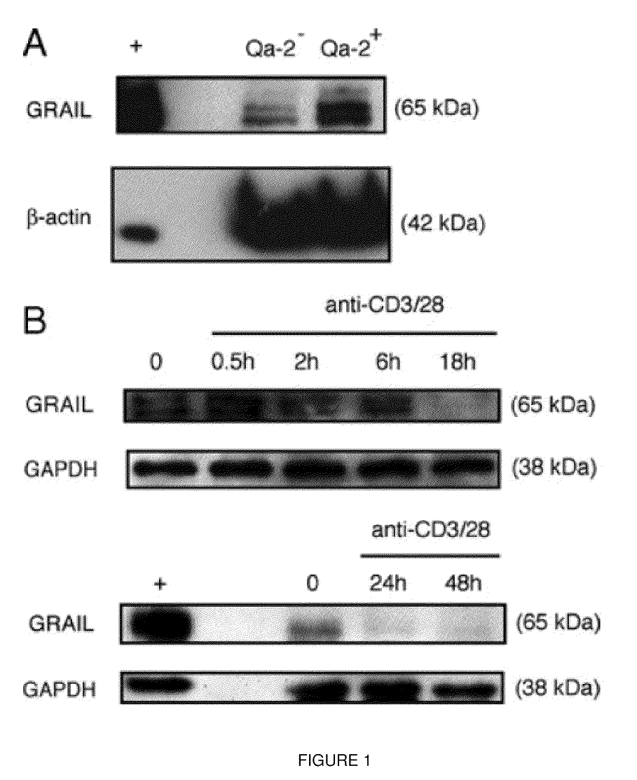

GRAIL is expressed in naive CD4 T cells and down-regulated during activation. To ask when and where GRAIL is initially expressed, we examined T cells from the thymus of BALB/c mice. We found that GRAIL was expressed abundantly in Qa-2+ late-stage, and less so in Qa-2− early-stage, single-positive CD4 T cells (FIG. 1A) but not in earlier-stage thymocytes. Qa-2 is a nonpolymorphic MHC class I Ag that is expressed on the cell surface of all peripheral CD4 T cells and on the subset of mature single-positive CD4 T cells in the thymus primed for exit to the periphery. GRAIL protein is also present in peripheral naive mouse CD4 T cells isolated ex vivo but is lost within 18 h and absent for up to 48 h following anti-CD3/anti-CD28 (anti-CD3/28) activation of these cells (FIG. 1B). These data show that late-stage, single-positive CD4 thymocytes and peripheral, naive CD4 T cells express GRAIL and that GRAIL expression is lost upon activation.

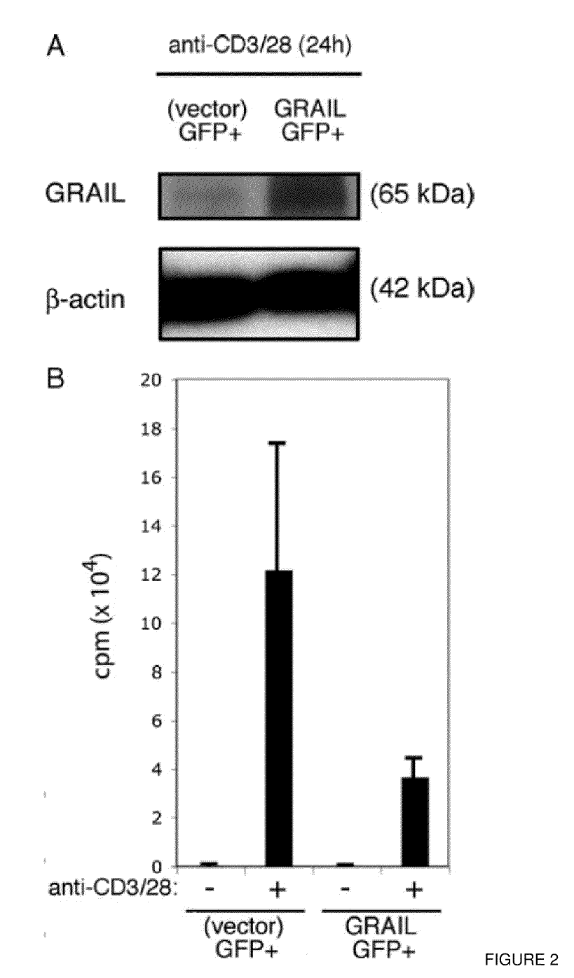

Down-regulation of GRAIL following stimulation of CD4 T cells is required for optimal proliferation. To demonstrate that the loss of GRAIL expression in naive CD4 T cells has a functional consequence, we used retroviral transduction of naive mouse CD4 T cells with GRAIL-expressing or control vector, both expressing GFP as a reporter, and sorted GFP+ cells for analysis (FIG. 1). Twenty-four hours following activation, when endogenous GRAIL is absent, immunoblots of cell lysates verified ectopic GRAIL expression in the CD4 T cells transduced with the GRAIL-expressing vector when compared with vector control transduced cells (FIG. 2A). As a consequence of maintaining transgenic GRAIL expression during anti-CD3/28 activation, proliferation of CD4 T cells transduced to express GRAIL was markedly inhibited compared with vector control-transduced CD4 T cells (FIG. 2B). Thus, GRAIL expressed in peripheral naive CD4 T cells maintains unresponsiveness, and its down-regulation is functionally required during T cell activation to allow proliferation.

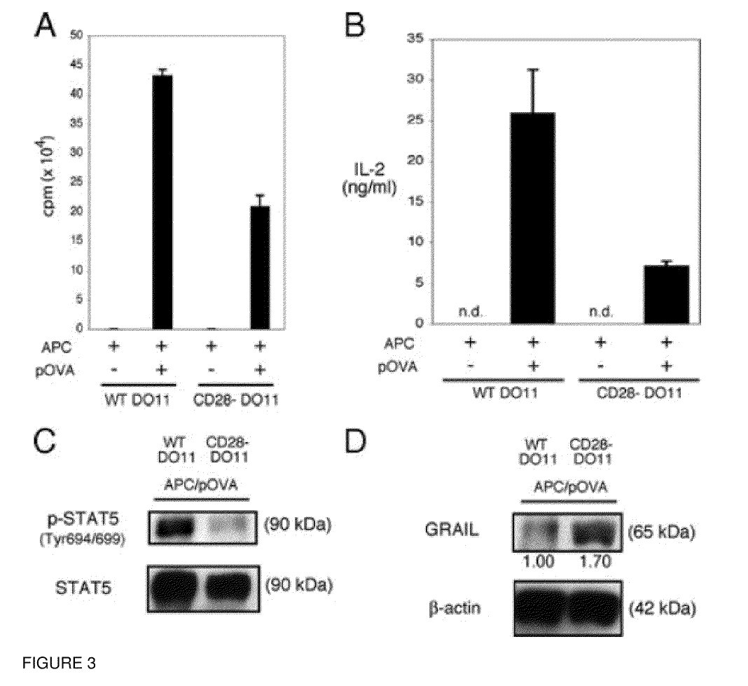

CD28 costimulation is required for GRAIL down-regulation, IL-2 production, and CD4 T cell proliferation. Successful activation of naive CD4 T cells requires both productive TCR/CD3 engagement and CD28 costimulation. We confirmed this using naive CD4 T cells from DO11 CD28^{+/+} or DO11 CD28^{-/-} transgenic BALB/c mice. As expected, in response to Ag-pulsed APC, proliferation of DO11 CD28^{-/-} CD4 T cells was diminished when compared with that of DO11 CD28^{+/±} CD4 T cells (FIG. 3A). In the absence of CD28 costimulation, IL-2 production was diminished (FIG. 3B), and this diminished production of IL-2 resulted in decreased IL-2R signaling as demonstrated by reduced STAT5 phosphorylation (FIG. 3C). When GRAIL was examined following activation of naive DO11 CD28^{+/+} or DO11 CD28^{-/-} CD4 T cells, GRAIL expression was markedly diminished in the activated DO11 CD28^{+/+} cells, while, in comparison, GRAIL expression was maintained in CD4 T cells from the DO11 CD28^{-/-} mice (FIG. 3D). This follows other reports implicating CD28 costimulation during T cell activation as a necessary component in triggering the loss of inhibitory E3 ubiquitin ligases, in this case leading to GRAIL degradation.

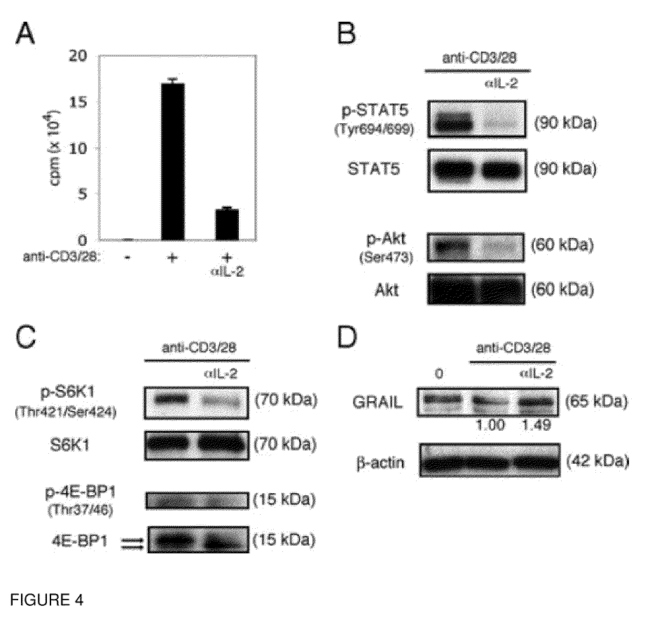

IL-2R signaling down-regulates GRAIL, allowing CD4 T cell proliferation. An important function of TCR/CD3 engagement is up-regulation of the IL-2Rα-chain (CD25) to form the high-affinity heterotrimeric IL-2R. CD28 costimulation triggers CD4 T cell production of the growth-promoting cytokine IL-2. Thus, following full activation of CD4 T cells, in an autocrine or paracrine fashion, IL-2 engages the high-affinity IL-2R and uses STAT5 and Akt signaling to drive CD4 T cell proliferation and differentiation. As the absence of CD28 costimulation led to diminished IL-2 production and IL-2R signaling, we investigated the role of IL-2 in modulating GRAIL expression. Blocking IL-2R engagement during mouse naive CD4 T cell activation, using a neutralizing anti-IL-2 Ab, inhibited CD4 T cell proliferation (FIG. 4A) and blocked phosphorylation of STAT5 and Akt (FIG. 4B). As Akt phosphorylation was deficient in the absence of IL-2R signaling, we also observed diminished mTOR activity assessed by decreased phosphorylation of S6K1 and 4E-BP1 (FIG. 4C). This suggested as one possibility that inhibition of IL-2R signaling might result in maintenance of GRAIL expression that would inhibit CD4 T cell proliferation. In support of this possibility, GRAIL expression was maintained following naive CD4 T cell activation in the presence of anti-IL-2 (FIG. 4D). These data suggest that during naive CD4 T cell activation, IL-2 production and IL-2R engagement are necessary for GRAIL degradation.

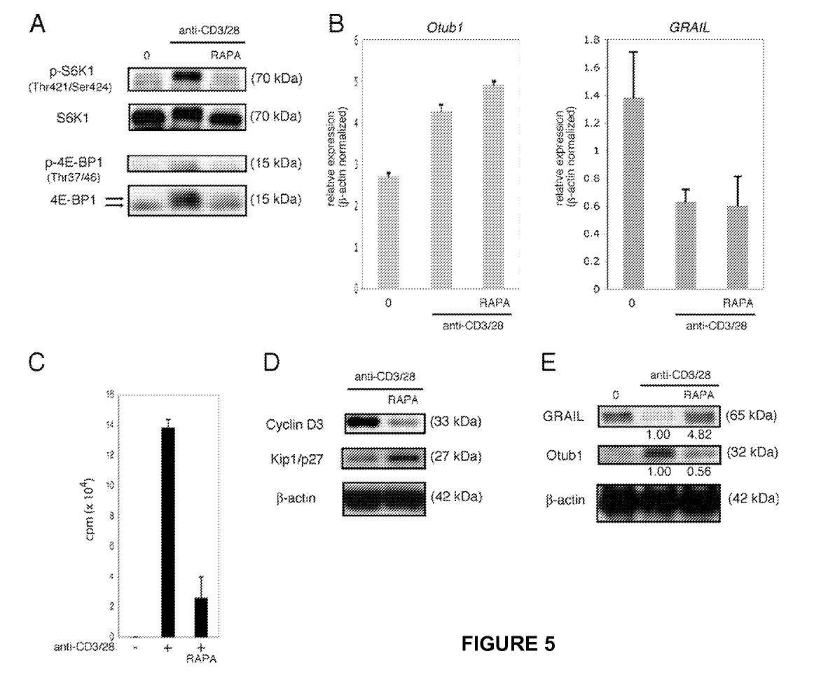

mTOR inhibition prevents Otub1 protein expression and maintains GRAIL, resulting in diminished cell proliferation. mTOR is a signal transduction kinase whose phosphorylation and subsequent kinase activity promote both overall protein translation and augment specific protein translation of a subset of mRNA. mTOR phosphorylation depends on the input of growth factor signals received by the cell, and mTOR kinase activity can be monitored by phosphorylation of S6K1 and 4E-BP1. When T cells are productively stimulated, mTOR is activated through phosphorylation via a pathway involving phosphorylated Akt. Activated mTOR phosphorylates its targets S6K1 and 4E-BP1. mTOR activated through phosphorylation is involved in T cell activation and trafficking. Because CD28 costimulation drives production of IL-2, and IL-2R engagement and signaling are both important growth signals for CD4 T cells and activate Akt and mTOR, we directly assessed the involvement of mTOR by using the small molecule mTOR inhibitor, rapamycin. As expected, treatment with rapamycin during activation of mouse naïve CD4 T cells resulted in the inhibition of mTOR activity as demonstrated by lack of phosphorylation of S6K1 and 4E-BP1 (FIG. 5A). As inhibition of IL-2R signaling during activation coincided with diminished mTOR activity and maintenance of GRAIL expression, we reasoned that rapamycin blockade of mTOR activity might lead to a decrease in Otub1 expression, accounting for the continued presence of GRAIL. When assessing Otub1 mRNA levels during mouse naive CD4 T cell activation in the presence or absence of mTOR inhibition, however, there was no demonstrable correlation between Otub1 mRNA levels and GRAIL expression. Activation of the T cells increased the level of Otub1 mRNA with or without the addition of rapamycin (FIG. 5B, left panel), whereas GRAIL mRNA levels were decreased regardless of the absence or presence of rapamycin (FIG. 5B, right panel). However, GRAIL protein was shown to be absent when mTOR was active and present when mTOR was inactive, and direct inhibition of mTOR activity by rapamycin inhibited proliferation (FIG. 5C) and cell division (FIG. 2).

Expression of cyclin D3, a pro-cell cycle molecule, and p27/Kip1, an anti-cell cycle molecule, were diminished and increased, respectively, with rapamycin treatment (FIG. 5D). These discordant results were resolved by investigating Otub1 protein expression levels following rapamycin treatment. We had previously demonstrated that human Otub1 protein leads to degradation of human GRAIL protein, and in a similar manner, murine Otub1 protein leads to murine GRAIL protein degradation (FIG. 3).

Ex vivo-isolated naive CD4 T cells express no Otub1 protein, thus allowing GRAIL expression (FIG. 5E, first lane), despite the presence of detectable Otub1 mRNA as mTOR-mediated protein translation is inactive in these cells. Upon stimulation, mTOR is activated and Otub1 protein is expressed, leading to the degradation of GRAIL (FIG. 5E, second lane). When mTOR is inhibited by rapamycin treatment during stimulation, although Otub1 mRNA is up-regulated, Otub1 protein is not expressed, allowing GRAIL to be maintained (FIG. 5E, third lane). Although regulation of GRAIL mRNA levels may be involved in GRAIL protein down-regulation upon stimulation, protein expression is dominantly influenced by regulatory factors at the protein level. These findings show that mTOR activation is required for naive CD4 T cell proliferation by permitting Otub1 protein expression and GRAIL degradation. Consistent with the mTOR function of promoting mRNA translation through activation of its downstream targets S6K1 and 4E-BP1, these results support our hypothesis that regulation of GRAIL expression by mTOR is at the Otub1 protein expression level.

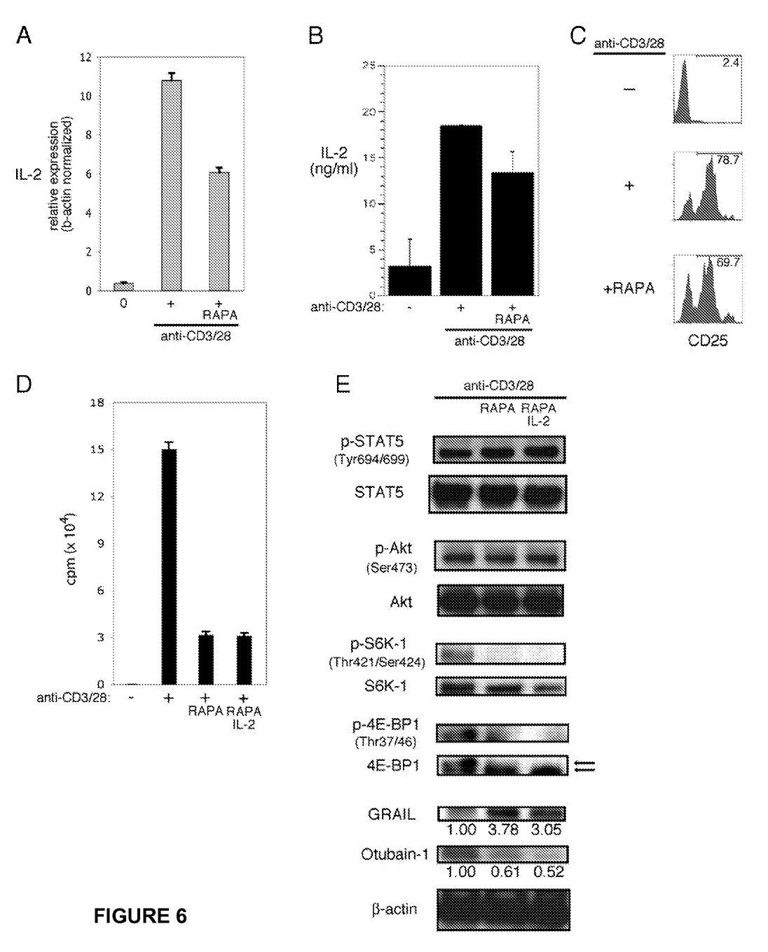

mTOR is the downstream critical component of IL-2R signaling regulating Otub1 and GRAIL. Inhibition of mTOR activity is sufficient to block Otub1 protein expression and maintain GRAIL, resulting in diminished cell proliferation. However, direct inhibition of mTOR activity by rapamycin may have been indirectly due to diminished IL-2 production and IL-2R signaling. As evidence of this possibility, we observed decreased IL-2 mRNA levels (FIG. 6A) and IL-2 production (FIG. 6B) by naive CD4 T cells stimulated in the presence of rapamycin. This diminution of IL-2 may have had a quantitative effect on IL-2R signaling or may have arisen through a delay in IL-2 production during an early critical phase, subsequently affecting GRAIL expression and proliferation.

The activation-induced component of the high-affinity IL-2R, CD25 (IL-2Rα), was increased to similar levels when stimulated in the presence or absence of rapamycin (FIG. 6C). This indiscriminate up-regulation of CD25 enables potentially equivalent IL-2R signaling; however, diminished IL-2 production in the presence of rapamycin may still have accounted for the observed differences. We reasoned that addition of exogenous IL-2 at the start of activation could compensate for either diminished or delayed IL-2 production in the presence of rapamycin; however, addition of exogenous IL-2 did not overcome the rapamycin induced inhibition of cell proliferation (FIG. 6D). Following exogenous IL-2 addition, rapamycin did not inhibit phosphorylation of STAT5 or Akt but specifically inhibited mTOR activity as demonstrated by decreased phosphorylation of S6K1 and 4E-BP1, reduced Otub1 protein, and maintenance of GRAIL (FIG. 6E). The intact phosphorylation of Akt was seen at both Ser473 and Thr308, suggesting the absence of any secondary effects by rapamycin inhibition of mTOR on the ability of the T cells to activate Akt. Thus, the critical component of IL-2R signaling regulating Otub1 and GRAIL, and their subsequent effects on proliferation, appears to be mTOR. Inhibition of mTOR, even in the presence of phosphorylated STAT5 and Akt, blocked Otub1 protein expression and maintained GRAIL expression, resulting in the inhibition of cell proliferation.

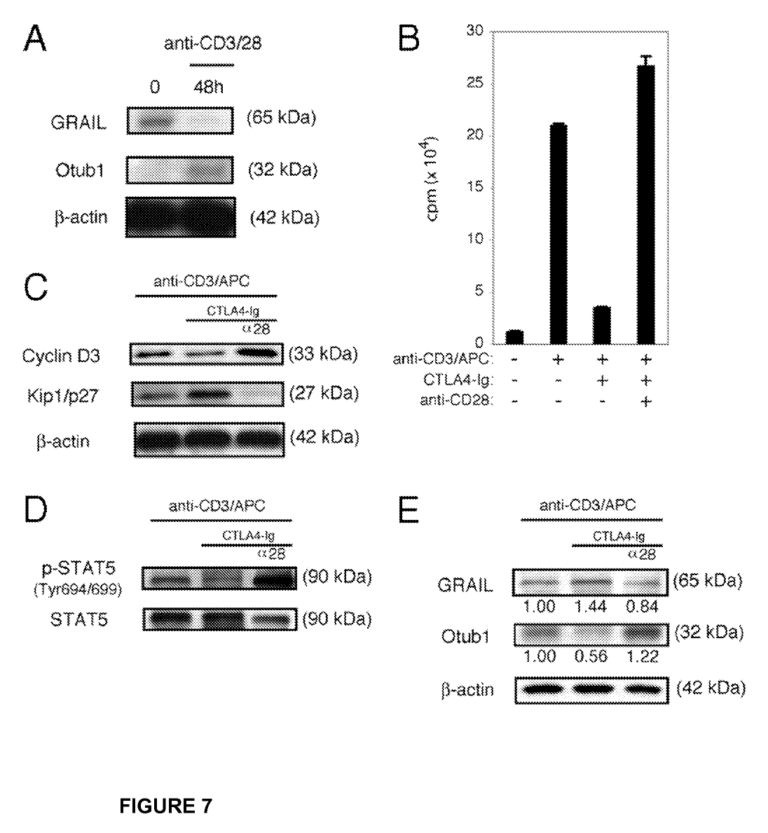

Human naive CD4 T cells require CD28 co-stimulation and IL-2R signaling during stimulation to down-regulate GRAIL. GRAIL and Otub1 interactions were originally identified in a yeast two-hybrid screen using a human genomic library. Recent studies have demonstrated that GRAIL expression is associated with anergy and inhibition of proliferation during human CD4 T cell activation. Thus, we asked whether human GRAIL and Otub1 regulation used the same pathway as that described in mouse naive CD4 T cells by examining activation of human naïve CD4⁺ CD45RA⁺ T cells (FIG. 4). We initially demonstrated that GRAIL was expressed in human naive CD4⁺ CD45RA⁺ T cells isolated ex vivo and, following stimulation using plate-bound anti-CD3 and APC (added to supply B7 for CD28 costimulation; FIG. 5i), GRAIL expression was lost, and Otub1 protein expression was observed (FIG. 7A). The proliferation of human naive CD4+ CD45RA+ T cells, activated in this manner, was inhibited when CTLA4-Ig was included in the culture, but proliferation was restored if agonist anti-CD28 Ab was added to the CTLA4-Ig containing cultures (FIG. 7B and FIG. 5ii). CTLA4-Ig inhibition of CD28 costimulation resulted in diminished cyclin D3 and increased Kip1/p27, which was also reversed by the addition of agonist anti-CD28 Ab (FIG. 7C).

When CD28 costimulation was blocked by CTLA4-Ig treatment, IL-2R signaling was impaired as seen by a decrease in phosphorylation of STAT5 and reversed by the addition of agonist anti-CD28 Ab (FIG. 7D). Addition of exogenous IL-2 to CTLA4-Ig treatment reversed the inhibition of proliferation (FIG. 6), suggesting that the lack of CD28 costimulation did not inhibit IL-2R up-regulation but that IL-2 production was impaired. The inhibition of CD28 costimulation by CTLA4-Ig treatment blocked Otub1 expression and sustained GRAIL expression, and this effect could be reversed by the addition of agonist anti-CD28 Ab (FIG. 7E). These findings in human naive CD4 T cells mirror our findings in mouse naive CD4 T cells. We conclude that CD28 costimulation, and resultant IL-2 production and IL-2R signaling, are important events regulating Otub1 and GRAIL expression and proliferation in human as well as mouse naive CD4 T cells.

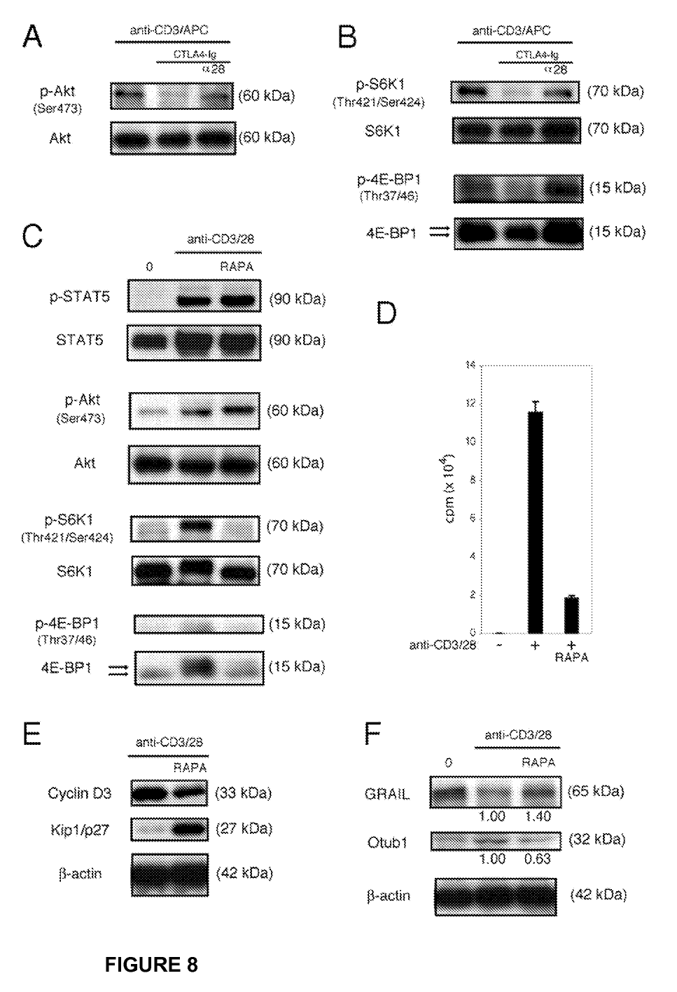

Human naive CD4 T cells require mTOR activation to allow Otub1 protein expression and GRAIL degradation. Since human naive CD4 T cells require CD28 costimulation and IL-2R signaling to modulate GRAIL expression, we reasoned that the mTOR pathway might also control human Otub1 and GRAIL expression. CD28 costimulation blockade of human naive CD4 T cell activation resulted in decreased phosphorylation of Akt, an upstream component within the mTOR activation pathway (FIG. 8A). In examining mTOR activity, phosphorylation of S6K1 and 4E-BP1 were both down-regulated in the presence of CTLA4-Ig and restored with direct agonist anti-CD28 Ab costimulation (FIG. 8B). Treatment of human naive CD4 T cells with rapamycin did not affect the phosphorylation of STAT5 or Akt but resulted in the inhibition of mTOR activity as measured by decreased phosphorylation of S6K1 and 4E-BP1 (FIG. 8C), similar to results seen in mouse CD4 T cells. Rapamycin treatment inhibited human naïve CD4 T cell proliferation (FIG. 8D) and resulted in decreased Cyclin D3 and increased Kip1/p27 levels (FIG. 8E). Human naive CD4 T cells activated in the presence of rapamycin failed to up-regulate Otub1 protein and maintained GRAIL expression (FIG. 8F). These results are identical to the effects seen in mouse naive CD4 T cells.

### Discussion

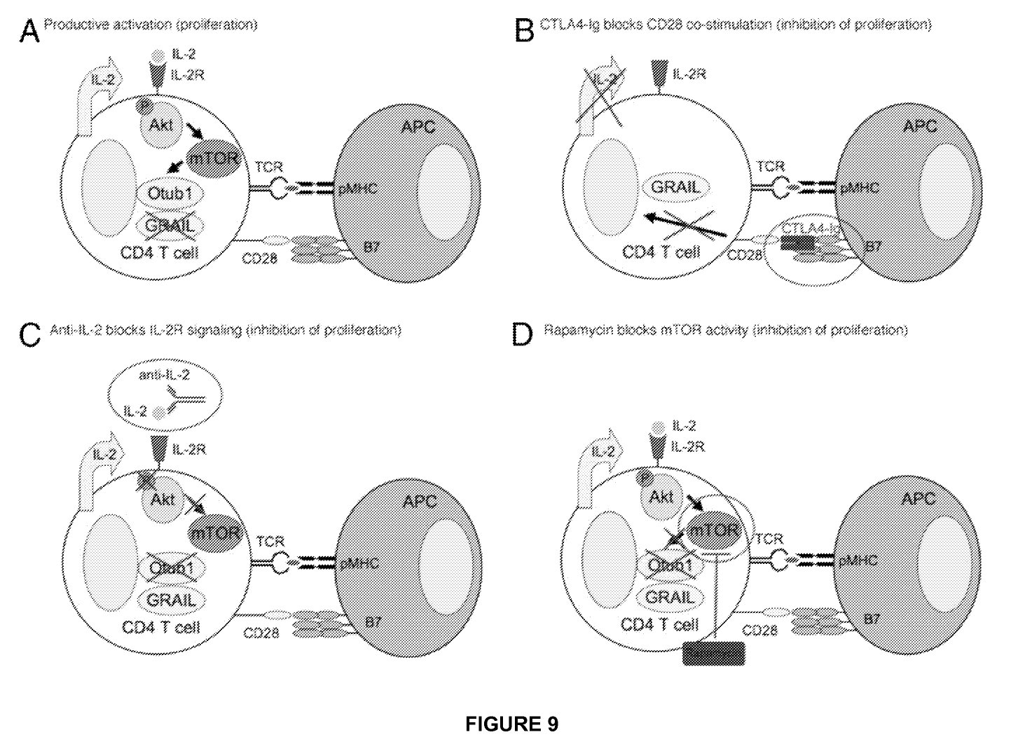

GRAIL is expressed in human and mouse naive CD4 T cells, and its degradation following TCR/CD3 engagement and costimulation is required for proliferation. These data demonstrate a role for GRAIL in controlling naive CD4 T cell activation and proliferation in addition to GRAIL's role in the induction and maintenance of anergy. As demonstrated in these studies, not only TCR/CD3 engagement and CD28 costimulation are required for full activation, IL-2 production and IL-2R signaling are also necessary to allow proliferation. Phosphorylation of Akt following IL-2R engagement drives mTOR activation leading to Otub1 protein expression, degradation of GRAIL, and T cell proliferation. These data demonstrate a pathway of GRAIL regulation that links critical components of CD4 T cell stimulation to CD4 T cell proliferation. Interference in this pathway highlights the potential importance of this pathway in peripheral T cell tolerance and may suggest new targets for immunotherapeutics (FIG. 9).

Our results link TCR/CD3 engagement and CD28 costimulation with IL-2 production and IL-2R signaling to activation of mTOR kinase that is required for activation induced proliferation of human and mouse naive CD4 T cells. Our studies highlight the importance of IL-2R signaling in sustaining mTOR activation during naive CD4 T cell activation. However, we also found that at early times (10 min to 1 h) following activation by CD3 and CD28 signaling, Akt is phosphorylated even in the presence of anti-IL-2 Abs, resulting in mTOR activation independent of IL-2R signaling (our unpublished observations) in agreement with previous reports. This discrepancy is resolved by differentiating the IL-2R signaling requirement at different time points following naive CD4 T cell activation. At later times (24-72 h) following activation, IL-2R signaling was required for sustained mTOR activity as anti-IL-2 Abs blocked phosphorylation of Akt and mTOR activation at these later time points (FIGS. 4, B and C), resulting in the sustained presence of GRAIL (FIG. 4D) and decreased proliferation (FIG. 4A).

Previous studies have implicated S6K1 regulation by mTOR in CD4 T cell activation, identifying a role for this pathway in directing mTOR-dependent protein translation. In this study, we demonstrate that naive CD4 T cells also regulate 4E-BP1 through the mTOR-dependent pathway via phosphorylation on Thr^{37/46}.

Phosphorylation of 4E-BP1 leads to its dissociation from eIF4E, allowing active eIF4E to bind eIF4G during translation initiation complex formation. A functional consequence attributed to active eIF4E is preferential translation of specific mRNAs normally translated into protein at low or absent rates. The phosphorylation of 4E-BP1, and subsequent activation of eIF4E, may allow protein translation of a subset of mRNAs important for T cell activation. We propose that Otub1 mRNA is under such regulation as its protein expression does not appear to be mediated through changes in mRNA transcript levels yet is sensitive to mTOR inhibition. The therapeutic effects of rapamycin in the inhibition of CD4 T cell activation and proliferation may be due not only to decreased overall protein translation but also to prevention of translation of a subset of mRNAs critical for successful activation.

This study is the first demonstration that endogenous GRAIL protein regulation in primary human and mouse naive CD4 T cells plays an important role in controlling T cell activation and proliferation. In mice, GRAIL expression can be traced to Qa-2+ CD4 single-positive thymocytes poised for export to the periphery; thus, GRAIL expression may be an important component of peripheral tolerance in naive CD4 T cells, in addition to its role in CD4 T cell anergy. Qa-2+ CD4 single-positive thymocytes, but not earlier stage thymocytes, respond to TCR ligation in a manner similar to peripheral CD4 T cells. The observations of GRAIL expression in Qa-2+ CD4 single-positive thymocytes and expression in peripheral naive CD4 T cells suggest a possible role for GRAIL in CD4 T cell tolerance to TCR self-peptide/MHC encountered during the transition from the thymus to the peripheral environment. TCR engagement of self selecting-peptide/MHC needs to remain a nonresponsive event for the naive CD4 T cell, and yet TCR engagement is necessary for maintaining their survival and keeping them poised for potential activation by non-self.

When foreign Ag is presented as non-self-peptide in the context of MHC class II, the increased affinity/avidity of the TCR engagement, as well as the presence of danger-induced APC costimulatory signals following B7-CD28 ligation, breaks the quiescent state of the naïve CD4 T cell that these data suggest is maintained by GRAIL. IL-2 signals through the IL-2R on CD4 T cells via mTOR to ensure GRAIL degradation to allow proliferation. Thus, maintenance of GRAIL serves to preserve quiescence of naive CD4 T cells and its down-regulation is required to allow proliferation. Anergic CD4 T cells express multiple E3 ubiquitin ligases, suggesting possible unique roles in maintaining cellular nonresponsiveness. The differential expression of these E3 ubiquitin ligases in primary CD4 T cells during quiescence and activation may provide insights into further elucidation of their functions in peripheral T cell tolerance. We found that while GRAIL was present in naive quiescent CD4 T cells and down-regulated upon activation, by contrast, Cbl-b was expressed at low levels in naïve quiescent CD4 T cells and up-regulated upon activation. Another group has recently also reported on the observed Cbl-b up-regulation upon activation in primary CD4 T cells. Their findings suggest that Cbl-b acts to limit CD4 T cell proliferation following TCR and CD28 activation through Cbl-b ubiquitination and degradation of phospholipase Cy and PI3K. Cbl-b decrease of PI3K expression diminishes downstream phosphorylation of ERK and Akt. We proposed that GRAIL and Cbl-b both serve to counteract CD4 T cell activation, however, at different stages. GRAIL, by maintaining quiescence in the absence of CD28 costimulation, and Cbl-b, by dampening proliferation of activated cells. GRAIL and Cbl-b may be mechanistically linked through Cbl-b down-regulation of Akt phosphorylation. A decrease in Akt phosphorylation would decrease mTOR activation, abrogating Otub1 protein expression and thus resulting in the reexpression of GRAIL and inhibition of cell proliferation. In this regard, we found that human and mouse naive CD4 T cells activated and subsequently rested eventually diminished their levels of phosphorylated Akt, S6K1, and 4E-BP1. The return of these cells to a nonproliferating quiescent state was correlated with the re-expression of GRAIL. Reactivation by TCR/CD3 and CD28 stimulation again led to Otub1 protein expression and down-regulation of GRAIL before cell proliferation.

NOD mice serve as a murine model of human type 1 diabetes with increasing incidence of hyperglycemia with age. The disease process is thought to occur initially through autoimmune T cell activation, possibly in the pancreatic lymph node, followed by inflammation of the islets of langerhans (insulitis) that, at about 12 wk of age, leads to islet β-cell destruction and resultant hyperglycemia. In search of genes differentially expressed during disease initiation and progression, we examined pancreatic lymph nodes from NOD and disease-resistant NOD.B10 (H-2b) congenic mice. We conducted genome-wide analyses of gene expression using microarrays comparing NOD vs NOD.B10 pancreatic lymph node RNA. At certain ages, including 12 wk, GRAIL expression was decreased in pancreatic lymph nodes of NOD mice compared with NOD.B10 mice (FIG. 7A). This differential GRAIL expression was verified by quantitative PCR of pancreatic lymph node RNA samples from multiple 12-wk-old NOD and NOD.B10 mice (FIG. 7B).

Our findings describe a peripheral tolerance role for GRAIL on naive CD4 T cells in vivo, which may be lost during NOD disease pathogenesis. In a study of primate HIV infection, GRAIL was up-regulated in anergic CD4 T cells isolated from disease-susceptible SIV-infected rhesus macaques, whereas SIV-resistant sooty mangabey primates showed no increase in GRAIL. A role for GRAIL in human disease was recently demonstrated in patients successfully treated for ulcerative colitis: patients in remission expressed higher levels of GRAIL in CD4 T cells vs patients with ongoing disease or normal controls. These studies and the findings reported above suggest that regulation of GRAIL and Otub1 plays an important role in peripheral tolerance.

### Example 2

### Inhibition of Antigen Sensitized Effector T Cells

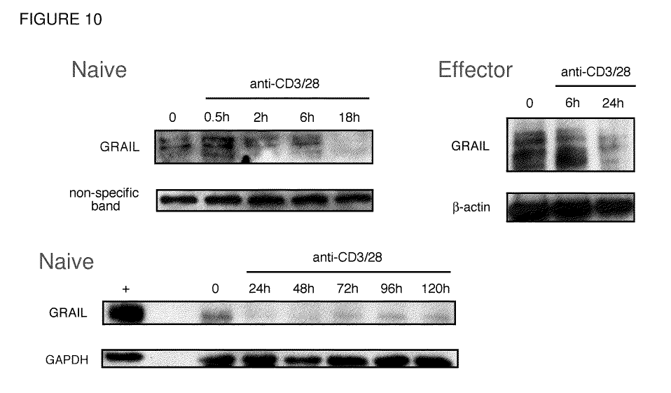

GRAIL is expressed during quiescence, lost upon activation. Naïve CD4 T cells were isolated from mouse and stimulated with anti-CD3 and anti-CD28 antibodies in vitro for the indicated number of hours (FIG. 10). Protein was collected and run by Western blot probing for GRAIL and β-actin. Effector CD4 T cells were made by first isolating from naïve mice, then stimulating with anti-CD3/28 for 3 days, split off and rested for 2 more days, then washed and put in fresh media for 2 additional days. Live CD4 T cells were then collected, shown to be at rest, and then activated in the same manner as described above. Protein samples were isolated and probed with anti-GRAII antibody as described in Example 1. The data demonstrate that GRAIL is last after activation of both naïve and effector T cells.

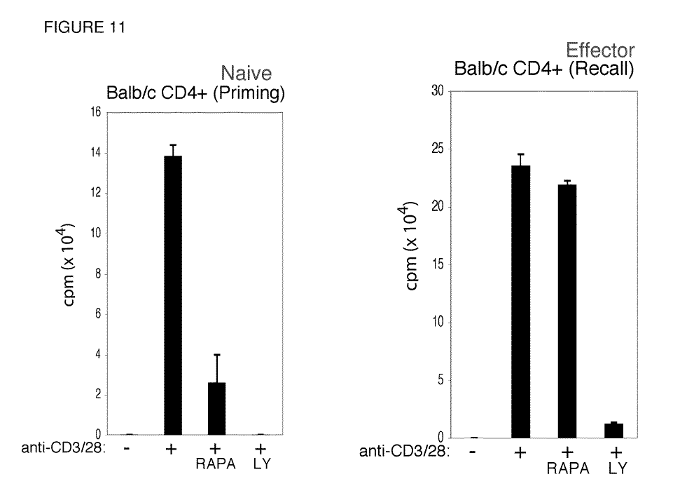

Shown in FIG. 11, naïve or effector CD4 cells were isolated as described above. The cells were assayed in a standard proliferation assay as described in Example 1. It was found that naïve T cells could be inhibited with rapamycin alone, but effector cells are not. The LY294002 drug inhibits PI3K, impinging on multiple pathways including both mTOR and MAPK/ERK, and thus the single agent blocks both pathways.

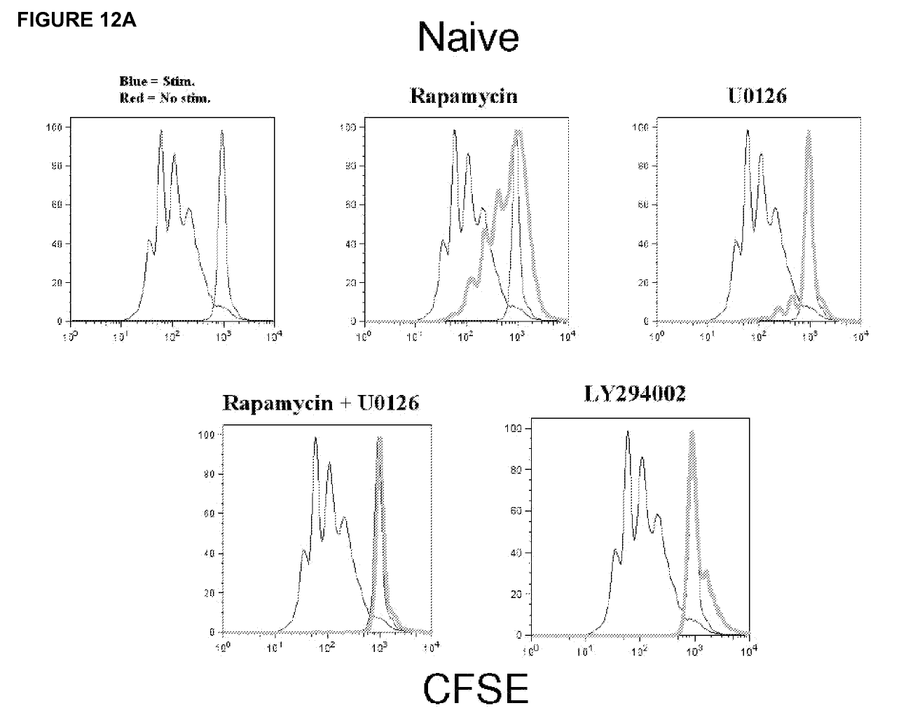

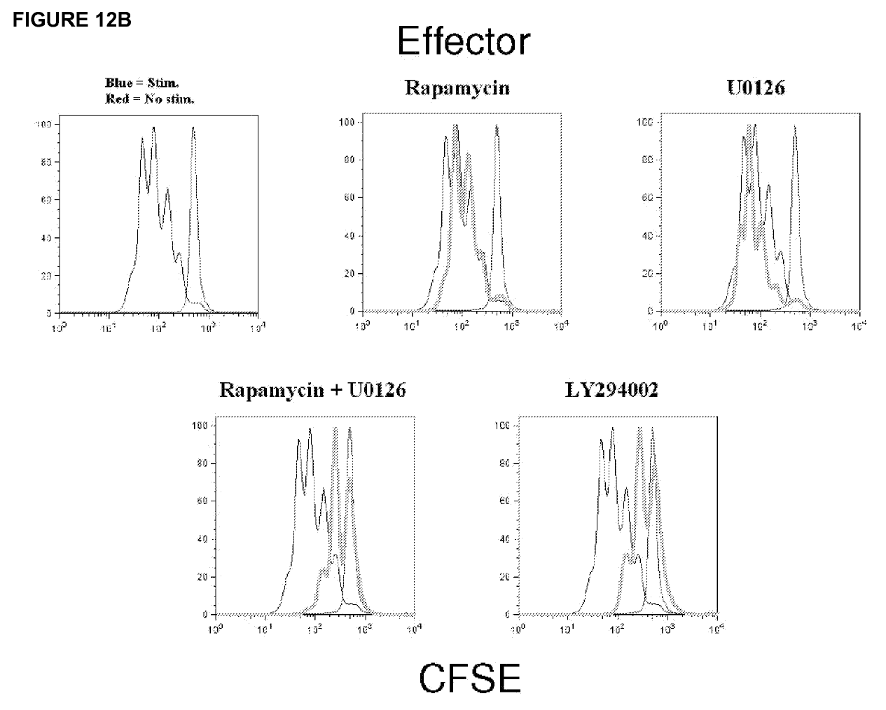

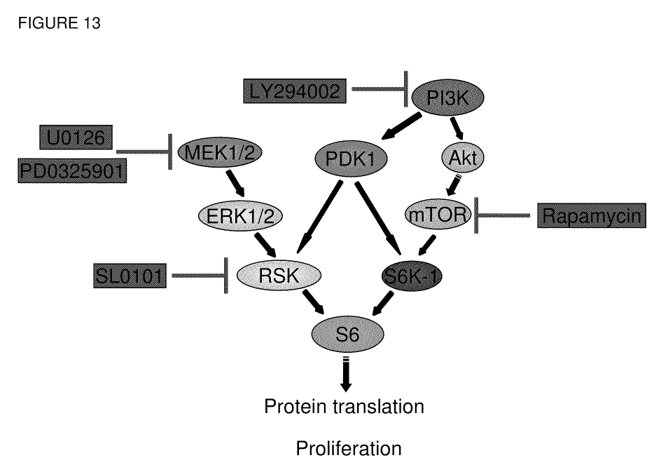

Shown in FIG. 12A, naïve CD4 T cells were labeled with CFSE, a dye that dilutes out with successive cell divisions. The naïve CD4 T cells were stimulated with anti-CD3/28 and the indicated drug(s) and CFSE profiles assessed after 3 days. Here, Rapamycin or U0126 alone inhibits proliferation (mTOR or MAPK/ERK blockade, LY294002 blocks both pathways). As these are naïve CD4 T cells, there is also an inhibition of proliferation with CTLA4-Ig and anti-IL-2 (mTOR pathways inhibitors). A similar experiment was performed with effector CD4 T cells, prepared as described above. With these cells, rapamycin or U0126 alone does not inhibit proliferation (mTOR or MAPK/ERK blockade) but the combination of both drugs or LY294002 is effective. Similar results were obtained with PD0325901 (another MEK inhibitor) and SL0101 (an RSK inhibitor downstream of MEK within the MAPK/ERK pathway) to same effect: it does not work alone but does work in combination with Rapamycin.

Shown in FIG. 12C, a western blot of effector T cells was done. When stimulated with anti-CD3/28, effector CD4 T cells degrade GRAIL to allow for proliferation. Use of Rapamycin or U0126 alone does not lead to GRAIL retention, still allowing the cells to proliferate. Only through the combination of Rapamycin and U0126 is GRAIL maintained, thus inhibiting proliferation in effector CD4 T cells.

Similar results were obtained with isolated human effector T cells.

### Example 3

Collagen induced arthritis was induced in mice according to established protocols, as described by Yaghoubi et al. (2009) J Biomed Opt. 12(6):064025; Tarner et al. (2006) Autoimmun Rev. 5(2):148-52; Tarner et al. (2003) Ann NY Acad. Sci. 2003 September; 998:512-9; Smith et al. (2003) Gene Ther. 10(15):1248-57, each herein specifically incorporated by reference.

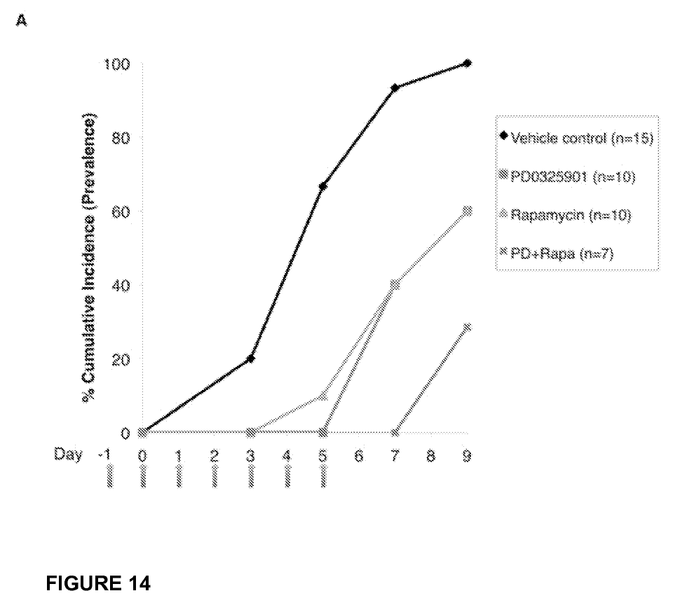

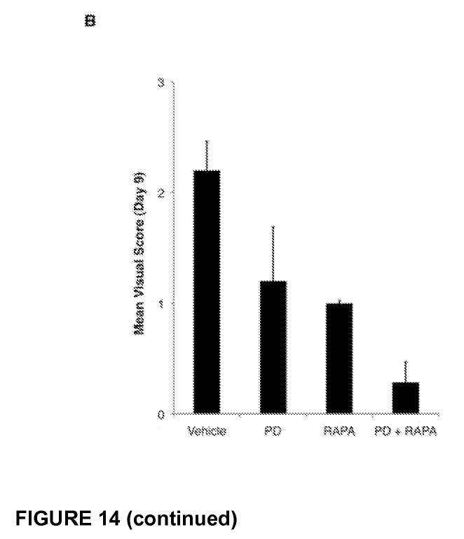

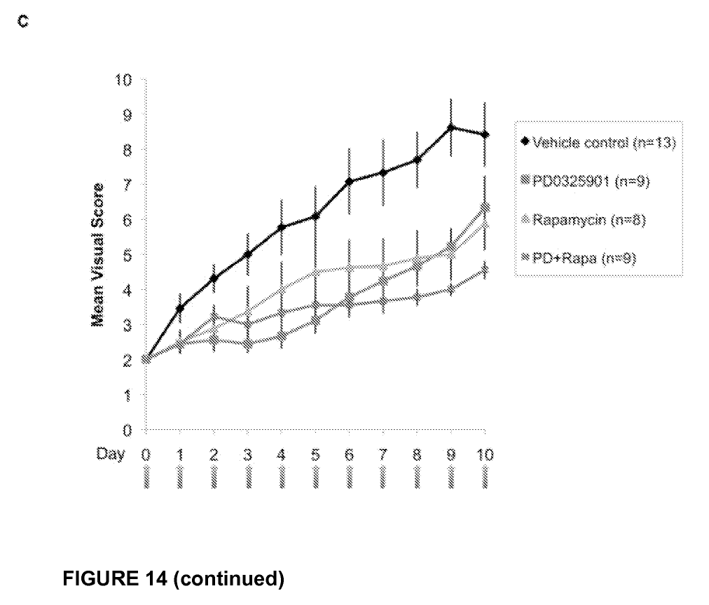

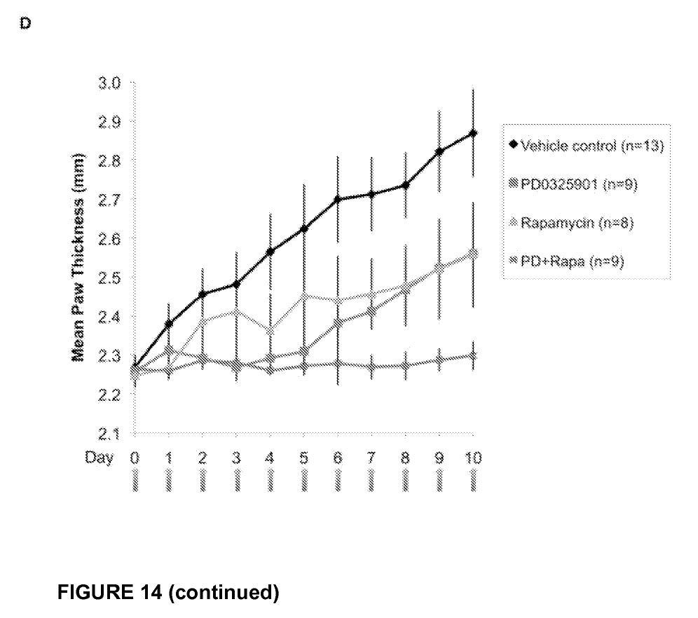

As shown in FIG. 14, treatment with the combined agents as set forth herein, including a combination of rapamycin and PD0325901 (N-(2,3-dihydroxypropoxy)-3,4-difluoro-2-(2-fluoro-4-iodophenylamino)benzamide; see Bain, J. et al., Biochem. J. 408:297-315 (2007)). The data show that the combined treatment provides for a lower prevalence of incidence, a lower mean visual score, and a reduced paw thickness when compared to either of the agents delivered as a single entity.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for inactivating an antigen sensitized CD4⁺ effector T cell, the method comprising:
contacting said T cell with (i) an inhibitor of the mTOR pathway; and (ii) an inhibitor of the MEK pathway;
wherein said T cell in inactivated in responsiveness to antigen.

2. The method of claim 1, wherein the mTOR pathway inhibitor acts on mTOR.

3. The method of claim 2, wherein the mTOR pathway inhibitor is rapamycin.

4. The method of claim 1, wherein the mTOR pathway inhibitor is CTLA4-Ig.

5. The method of claim 1, wherein the mTOR pathway inhibitor is anti-IL-2.

6. The method of claim 1, wherein the MEK pathway inhibitor inhibits MEK.

7. The method of claim 1, wherein the MEK pathway inhibitor inhibits RSK.

8. The method of claim 1 wherein one or both of said inhibitors is an antisense or RNAi molecule.

9. The method of claim 1, wherein the combination of inhibitors provides for a synergistic decrease in responsiveness to antigen.

10. The method of claim 1, wherein the T cell is a human T cell.

11. The method of claim 1, wherein the T cell population is present in an in vitro culture.

12. The method of claim 1, wherein the T cell population is present in an animal.

13. The method of claim 12, wherein the animal is suffering from an autoimmune disease.

14. The method of claim 12 wherein the animal is a transplant recipient.
